SEQUENCE LISTING (142 sequences)

SEQ ID NO: 1 - DNA, 11 bp, Artificial Sequence
  Description of Artificial Sequence Synthetic oligonucleotide
acgacgtacgt

SEQ ID NO: 2 - DNA, 11 bp, Artificial Sequence
  Description of Artificial Sequence Synthetic oligonucleotide
acacgacgact

SEQ ID NO: 3 - DNA, 11 bp, Artificial Sequence
  Description of Artificial Sequence Synthetic oligonucleotide
acacgtagtat

SEQ ID NO: 4 - DNA, 11 bp, Artificial Sequence
  Description of Artificial Sequence Synthetic oligonucleotide
acactactcgt

SEQ ID NO: 5 - DNA, 11 bp, Artificial Sequence
  Description of Artificial Sequence Synthetic oligonucleotide
acgacacgtat

SEQ ID NO: 6 - DNA, 11 bp, Artificial Sequence
  Description of Artificial Sequence Synthetic oligonucleotide
acgagtagact

SEQ ID NO: 7 - DNA, 11 bp, Artificial Sequence
  Description of Artificial Sequence Synthetic oligonucleotide
acgcgtctagt

SEQ ID NO: 8 - DNA, 11 bp, Artificial Sequence
  Description of Artificial Sequence Synthetic oligonucleotide
acgtacacact

SEQ ID NO: 9 - DNA, 11 bp, Artificial Sequence
  Description of Artificial Sequence Synthetic oligonucleotide
acgtactgtgt

SEQ ID NO: 10 - DNA, 11 bp, Artificial Sequence
  Description of Artificial Sequence Synthetic oligonucleotide
acgtagatcgt

SEQ ID NO: 11 - DNA, 11 bp, Artificial Sequence
  Description of Artificial Sequence Synthetic oligonucleotide
actacgtctct

SEQ ID NO: 12 - DNA, 11 bp, Artificial Sequence
  Description of Artificial Sequence Synthetic oligonucleotide
actatacgagt

SEQ ID NO: 13 - DNA, 11 bp, Artificial Sequence
  Description of Artificial Sequence Synthetic oligonucleotide
actcgcgtcgt

SEQ ID NO: 14 - DNA, 11 bp, Artificial Sequence
  Description of Artificial Sequence Synthetic oligonucleotide
agactcgacgt

SEQ ID NO: 15 - DNA, 11 bp, Artificial Sequence
  Description of Artificial Sequence Synthetic oligonucleotide
agtacgagagt

SEQ ID NO: 16 - DNA, 11 bp, Artificial Sequence
  Description of Artificial Sequence Synthetic oligonucleotide
agtactactat

SEQ ID NO: 17 - DNA, 11 bp, Artificial Sequence
  Description of Artificial Sequence Synthetic oligonucleotide
agtagacgtct

SEQ ID NO: 18 - DNA, 11 bp, Artificial Sequence
  Description of Artificial Sequence Synthetic oligonucleotide
agtcgtacact

SEQ ID NO: 19 - DNA, 11 bp, Artificial Sequence
  Description of Artificial Sequence Synthetic oligonucleotide
agtgtagtagt

SEQ ID NO: 20 - DNA, 11 bp, Artificial Sequence
  Description of Artificial Sequence Synthetic oligonucleotide
atagtatacgt

SEQ ID NO: 21 - DNA, 11 bp, Artificial Sequence
  Description of Artificial Sequence Synthetic oligonucleotide
cagtacgtact

SEQ ID NO: 22 - DNA, 11 bp, Artificial Sequence
  Description of Artificial Sequence Synthetic oligonucleotide
cgacgacgcgt

SEQ ID NO: 23 - DNA, 11 bp, Artificial Sequence
  Description of Artificial Sequence Synthetic oligonucleotide
cgacgagtact

SEQ ID NO: 24 - DNA, 11 bp, Artificial Sequence
  Description of Artificial Sequence Synthetic oligonucleotide
cgatactacgt

SEQ ID NO: 25 - DNA, 11 bp, Artificial Sequence
  Description of Artificial Sequence Synthetic oligonucleotide
cgtacgtcgat

SEQ ID NO: 26 - DNA, 11 bp, Artificial Sequence
  Description of Artificial Sequence Synthetic oligonucleotide
ctactcgtagt

SEQ ID NO: 27 - DNA, 11 bp, Artificial Sequence
  Description of Artificial Sequence Synthetic oligonucleotide
gtacagtacgt

SEQ ID NO: 28 - DNA, 11 bp, Artificial Sequence
  Description of Artificial Sequence Synthetic oligonucleotide
gtcgtacgtat

SEQ ID NO: 29 - DNA, 11 bp, Artificial Sequence
  Description of Artificial Sequence Synthetic oligonucleotide
gtgtacgacgt

SEQ ID NO: 30 - DNA, 11 bp, Artificial Sequence
  Description of Artificial Sequence Synthetic oligonucleotide
acacagtgagt

SEQ ID NO: 31 - DNA, 11 bp, Artificial Sequence
  Description of Artificial Sequence Synthetic oligonucleotide
acactcatact

SEQ ID NO: 32 - DNA, 11 bp, Artificial Sequence
  Description of Artificial Sequence Synthetic oligonucleotide
acagacagcgt

SEQ ID NO: 33 - DNA, 11 bp, Artificial Sequence
  Description of Artificial Sequence Synthetic oligonucleotide
acagactatat

SEQ ID NO: 34 - DNA, 11 bp, Artificial Sequence
  Description of Artificial Sequence Synthetic oligonucleotide
acagagactct

SEQ ID NO: 35 - DNA, 11 bp, Artificial Sequence
  Description of Artificial Sequence Synthetic oligonucleotide
acagctcgtgt

SEQ ID NO: 36 - DNA, 11 bp, Artificial Sequence
  Description of Artificial Sequence Synthetic oligonucleotide
acagtgtcgat

SEQ ID NO: 37 - DNA, 11 bp, Artificial Sequence
  Description of Artificial Sequence Synthetic oligonucleotide
acgagcgcgct

SEQ ID NO: 38 - DNA, 11 bp, Artificial Sequence
  Description of Artificial Sequence Synthetic oligonucleotide
acgatgagtgt

SEQ ID NO: 39 - DNA, 11 bp, Artificial Sequence
  Description of Artificial Sequence Synthetic oligonucleotide
acgcgagagat

SEQ ID NO: 40 - DNA, 11 bp, Artificial Sequence
  Description of Artificial Sequence Synthetic oligonucleotide
acgctctctct

SEQ ID NO: 41 - DNA, 11 bp, Artificial Sequence
  Description of Artificial Sequence Synthetic oligonucleotide
acgtcgctgat

SEQ ID NO: 42 - DNA, 11 bp, Artificial Sequence
  Description of Artificial Sequence Synthetic oligonucleotide
acgtctagcat

SEQ ID NO: 43 - DNA, 11 bp, Artificial Sequence
  Description of Artificial Sequence Synthetic oligonucleotide
actagtgatat

SEQ ID NO: 44 - DNA, 11 bp, Artificial Sequence
  Description of Artificial Sequence Synthetic oligonucleotide
actcacactgt

SEQ ID NO: 45 - DNA, 11 bp, Artificial Sequence
  Description of Artificial Sequence Synthetic oligonucleotide
actcactagct

SEQ ID NO: 46 - DNA, 11 bp, Artificial Sequence
  Description of Artificial Sequence Synthetic oligonucleotide
actctatatat

SEQ ID NO: 47 - DNA, 11 bp, Artificial Sequence
  Description of Artificial Sequence Synthetic oligonucleotide
actgatctcgt

SEQ ID NO: 48 - DNA, 11 bp, Artificial Sequence
  Description of Artificial Sequence Synthetic oligonucleotide
actgctgtact

SEQ ID NO: 49 - DNA, 11 bp, Artificial Sequence
  Description of Artificial Sequence Synthetic oligonucleotide
actgtagcgct

SEQ ID NO: 50 - DNA, 11 bp, Artificial Sequence
  Description of Artificial Sequence Synthetic oligonucleotide
agacactcact

SEQ ID NO: 51 - DNA, 11 bp, Artificial Sequence
  Description of Artificial Sequence Synthetic oligonucleotide
agacatatagt

SEQ ID NO: 52 - DNA, 11 bp, Artificial Sequence
  Description of Artificial Sequence Synthetic oligonucleotide
agacgtgatct

SEQ ID NO: 53 - DNA, 11 bp, Artificial Sequence
  Description of Artificial Sequence Synthetic oligonucleotide
agagtacagat

SEQ ID NO: 54 - DNA, 11 bp, Artificial Sequence
  Description of Artificial Sequence Synthetic oligonucleotide
agagtatctct

SEQ ID NO: 55 - DNA, 11 bp, Artificial Sequence
  Description of Artificial Sequence Synthetic oligonucleotide
agatacgctgt

SEQ ID NO: 56 - DNA, 11 bp, Artificial Sequence
  Description of Artificial Sequence Synthetic oligonucleotide
agatctagtct

SEQ ID NO: 57 - DNA, 11 bp, Artificial Sequence
  Description of Artificial Sequence Synthetic oligonucleotide
agcagcgtagt

SEQ ID NO: 58 - DNA, 11 bp, Artificial Sequence
  Description of Artificial Sequence Synthetic oligonucleotide
agcgcacgagt

SEQ ID NO: 59 - DNA, 11 bp, Artificial Sequence
  Description of Artificial Sequence Synthetic oligonucleotide
agcgtgtgcgt

SEQ ID NO: 60 - DNA, 11 bp, Artificial Sequence
  Description of Artificial Sequence Synthetic oligonucleotide
agctagatact

SEQ ID NO: 61 - DNA, 11 bp, Artificial Sequence
  Description of Artificial Sequence Synthetic oligonucleotide
agctgtcgact

SEQ ID NO: 62 - DNA, 11 bp, Artificial Sequence
  Description of Artificial Sequence Synthetic oligonucleotide
agtatgcacgt

SEQ ID NO: 63 - DNA, 11 bp, Artificial Sequence
  Description of Artificial Sequence Synthetic oligonucleotide
agtcgcgctat

SEQ ID NO: 64 - DNA, 11 bp, Artificial Sequence
  Description of Artificial Sequence Synthetic oligonucleotide
agtctgtctgt

SEQ ID NO: 65 - DNA, 11 bp, Artificial Sequence
  Description of Artificial Sequence Synthetic oligonucleotide
atacacacgat

SEQ ID NO: 66 - DNA, 11 bp, Artificial Sequence
  Description of Artificial Sequence Synthetic oligonucleotide
atacgcgtgct

SEQ ID NO: 67 - DNA, 11 bp, Artificial Sequence
  Description of Artificial Sequence Synthetic oligonucleotide
atactagcact

SEQ ID NO: 68 - DNA, 11 bp, Artificial Sequence
  Description of Artificial Sequence Synthetic oligonucleotide
atagagctagt

SEQ ID NO: 69 - DNA, 11 bp, Artificial Sequence
  Description of Artificial Sequence Synthetic oligonucleotide
atatagagtat

SEQ ID NO: 70 - DNA, 11 bp, Artificial Sequence
  Description of Artificial Sequence Synthetic oligonucleotide
atcgctcacgt

SEQ ID NO: 71 - DNA, 11 bp, Artificial Sequence
  Description of Artificial Sequence Synthetic oligonucleotide
atcgtcagtct

SEQ ID NO: 72 - DNA, 11 bp, Artificial Sequence
  Description of Artificial Sequence Synthetic oligonucleotide
atctctcgtat

SEQ ID NO: 73 - DNA, 11 bp, Artificial Sequence
  Description of Artificial Sequence Synthetic oligonucleotide
atctgagacgt

SEQ ID NO: 74 - DNA, 11 bp, Artificial Sequence
  Description of Artificial Sequence Synthetic oligonucleotide
atgctacgtct

SEQ ID NO: 75 - DNA, 11 bp, Artificial Sequence
  Description of Artificial Sequence Synthetic oligonucleotide
atgtgactact

SEQ ID NO: 76 - DNA, 11 bp, Artificial Sequence
  Description of Artificial Sequence Synthetic oligonucleotide
cacgagacagt

SEQ ID NO: 77 - DNA, 11 bp, Artificial Sequence
  Description of Artificial Sequence Synthetic oligonucleotide
cacgcgagtct

SEQ ID NO: 78 - DNA, 11 bp, Artificial Sequence
  Description of Artificial Sequence Synthetic oligonucleotide
cacgctacgat

SEQ ID NO: 79 - DNA, 11 bp, Artificial Sequence
  Description of Artificial Sequence Synthetic oligonucleotide
cacgtgtatat

SEQ ID NO: 80 - DNA, 11 bp, Artificial Sequence
  Description of Artificial Sequence Synthetic oligonucleotide
cactacgatgt

SEQ ID NO: 81 - DNA, 11 bp, Artificial Sequence
  Description of Artificial Sequence Synthetic oligonucleotide
cactatactct

SEQ ID NO: 82 - DNA, 11 bp, Artificial Sequence
  Description of Artificial Sequence Synthetic oligonucleotide
cagcgtactgt

SEQ ID NO: 83 - DNA, 11 bp, Artificial Sequence
  Description of Artificial Sequence Synthetic oligonucleotide
cagtctctagt

SEQ ID NO: 84 - DNA, 11 bp, Artificial Sequence
  Description of Artificial Sequence Synthetic oligonucleotide
catagtcgcgt

SEQ ID NO: 85 - DNA, 11 bp, Artificial Sequence
  Description of Artificial Sequence Synthetic oligonucleotide
cgagacactat

SEQ ID NO: 86 - DNA, 11 bp, Artificial Sequence
  Description of Artificial Sequence Synthetic oligonucleotide
cgagagtgtgt

SEQ ID NO: 87 - DNA, 11 bp, Artificial Sequence
  Description of Artificial Sequence Synthetic oligonucleotide
cgagtcatcgt

SEQ ID NO: 88 - DNA, 11 bp, Artificial Sequence
  Description of Artificial Sequence Synthetic oligonucleotide
cgatcgtatat

SEQ ID NO: 89 - DNA, 11 bp, Artificial Sequence
  Description of Artificial Sequence Synthetic oligonucleotide
cgcagtacgct

SEQ ID NO: 90 - DNA, 11 bp, Artificial Sequence
  Description of Artificial Sequence Synthetic oligonucleotide
cgcgatcgtat

SEQ ID NO: 91 - DNA, 11 bp, Artificial Sequence
  Description of Artificial Sequence Synthetic oligonucleotide
cgcgctatact

SEQ ID NO: 92 - DNA, 11 bp, Artificial Sequence
  Description of Artificial Sequence Synthetic oligonucleotide
cgtacagatat

SEQ ID NO: 93 - DNA, 11 bp, Artificial Sequence
  Description of Artificial Sequence Synthetic oligonucleotide
cgtagctctct

SEQ ID NO: 94 - DNA, 11 bp, Artificial Sequence
  Description of Artificial Sequence Synthetic oligonucleotide
cgtatagtgct

SEQ ID NO: 95 - DNA, 11 bp, Artificial Sequence
  Description of Artificial Sequence Synthetic oligonucleotide
cgtcagcgact

SEQ ID NO: 96 - DNA, 11 bp, Artificial Sequence
  Description of Artificial Sequence Synthetic oligonucleotide
cgtcgcagtgt

SEQ ID NO: 97 - DNA, 11 bp, Artificial Sequence
  Description of Artificial Sequence Synthetic oligonucleotide
cgtctcacgat

SEQ ID NO: 98 - DNA, 11 bp, Artificial Sequence
  Description of Artificial Sequence Synthetic oligonucleotide
cgtgactcagt

SEQ ID NO: 99 - DNA, 11 bp, Artificial Sequence
  Description of Artificial Sequence Synthetic oligonucleotide
ctacacgctct

SEQ ID NO: 100 - DNA, 11 bp, Artificial Sequence
  Description of Artificial Sequence Synthetic oligonucleotide
ctacgatatgt

SEQ ID NO: 101 - DNA, 11 bp, Artificial Sequence
  Description of Artificial Sequence Synthetic oligonucleotide
ctagacagact

SEQ ID NO: 102 - DNA, 11 bp, Artificial Sequence
  Description of Artificial Sequence Synthetic oligonucleotide
ctagtactcat

SEQ ID NO: 103 - DNA, 11 bp, Artificial Sequence
  Description of Artificial Sequence Synthetic oligonucleotide
ctatatgtcgt

SEQ ID NO: 104 - DNA, 11 bp, Artificial Sequence
  Description of Artificial Sequence Synthetic oligonucleotide
ctatcgacact

SEQ ID NO: 105 - DNA, 11 bp, Artificial Sequence
  Description of Artificial Sequence Synthetic oligonucleotide
ctatgtagagt

SEQ ID NO: 106 - DNA, 11 bp, Artificial Sequence
  Description of Artificial Sequence Synthetic oligonucleotide
ctcacgtacat

SEQ ID NO: 107 - DNA, 11 bp, Artificial Sequence
  Description of Artificial Sequence Synthetic oligonucleotide
ctcgagtctct

SEQ ID NO: 108 - DNA, 11 bp, Artificial Sequence
  Description of Artificial Sequence Synthetic oligonucleotide
ctcgtcgagat

SEQ ID NO: 109 - DNA, 11 bp, Artificial Sequence
  Description of Artificial Sequence Synthetic oligonucleotide
ctctacagcgt

SEQ ID NO: 110 - DNA, 11 bp, Artificial Sequence
  Description of Artificial Sequence Synthetic oligonucleotide
ctgtcgtgcgt

SEQ ID NO: 111 - DNA, 11 bp, Artificial Sequence
  Description of Artificial Sequence Synthetic oligonucleotide
ctgtgacgtgt

SEQ ID NO: 112 - DNA, 11 bp, Artificial Sequence
  Description of Artificial Sequence Synthetic oligonucleotide
gacgctgtcgt

SEQ ID NO: 113 - DNA, 11 bp, Artificial Sequence
  Description of Artificial Sequence Synthetic oligonucleotide
gacgtatgact

SEQ ID NO: 114 - DNA, 11 bp, Artificial Sequence
  Description of Artificial Sequence Synthetic oligonucleotide
gactagctagt

SEQ ID NO: 115 - DNA, 11 bp, Artificial Sequence
  Description of Artificial Sequence Synthetic oligonucleotide
gagacgtcgct

SEQ ID NO: 116 - DNA, 11 bp, Artificial Sequence
  Description of Artificial Sequence Synthetic oligonucleotide
gagagagacgt

SEQ ID NO: 117 - DNA, 11 bp, Artificial Sequence
  Description of Artificial Sequence Synthetic oligonucleotide
gcgtagactat

SEQ ID NO: 118 - DNA, 11 bp, Artificial Sequence
  Description of Artificial Sequence Synthetic oligonucleotide
gcgtcgtgtct

SEQ ID NO: 119 - DNA, 11 bp, Artificial Sequence
  Description of Artificial Sequence Synthetic oligonucleotide
gctctctacgt

SEQ ID NO: 120 - DNA, 11 bp, Artificial Sequence
  Description of Artificial Sequence Synthetic oligonucleotide
gtacactgtat

SEQ ID NO: 121 - DNA, 11 bp, Artificial Sequence
  Description of Artificial Sequence Synthetic oligonucleotide
gtacgcgacat

SEQ ID NO: 122 - DNA, 11 bp, Artificial Sequence
  Description of Artificial Sequence Synthetic oligonucleotide
gtactatagat

SEQ ID NO: 123 - DNA, 11 bp, Artificial Sequence
  Description of Artificial Sequence Synthetic oligonucleotide
gtactgagtct

SEQ ID NO: 124 - DNA, 11 bp, Artificial Sequence
  Description of Artificial Sequence Synthetic oligonucleotide
gtagctagcgt

SEQ ID NO: 125 - DNA, 11 bp, Artificial Sequence
  Description of Artificial Sequence Synthetic oligonucleotide
gtagtcactgt

SEQ ID NO: 126 - DNA, 11 bp, Artificial Sequence
  Description of Artificial Sequence Synthetic oligonucleotide
gtagtgtcact

SEQ ID NO: 127 - DNA, 11 bp, Artificial Sequence
  Description of Artificial Sequence Synthetic oligonucleotide
gtatacatagt

SEQ ID NO: 128 - DNA, 11 bp, Artificial Sequence
  Description of Artificial Sequence Synthetic oligonucleotide
gtcatcgtcgt

SEQ ID NO: 129 - DNA, 11 bp, Artificial Sequence
  Description of Artificial Sequence Synthetic oligonucleotide
gtcgacacgct

SEQ ID NO: 130 - DNA, 11 bp, Artificial Sequence
  Description of Artificial Sequence Synthetic oligonucleotide
gtcgagtgagt

SEQ ID NO: 131 - DNA, 11 bp, Artificial Sequence
  Description of Artificial Sequence Synthetic oligonucleotide
gtctactatct

SEQ ID NO: 132 - DNA, 11 bp, Artificial Sequence
  Description of Artificial Sequence Synthetic oligonucleotide
gtgtctagact

SEQ ID NO: 133 - DNA, 11 bp, Artificial Sequence
  Description of Artificial Sequence Synthetic oligonucleotide
gtgtgtatcgt

SEQ ID NO: 134 - DNA, 32 bp, Artificial Sequence
  Description of Artificial Sequence Synthetic oligonucleotide
ctgagtcggagacaaggcacacaggggatagg

SEQ ID NO: 135 - DNA, 31 bp, Artificial Sequence
  Description of Artificial Sequence Synthetic oligonucleotide
ccatctcatccctgcgtgtctccgactcagt

SEQ ID NO: 136 - DNA, 33 bp, Artificial Sequence
  Description of Artificial Sequence Synthetic oligonucleotide
actgagtcggagacaaggcacacaggggatagg

SEQ ID NO: 137 - DNA, 32 bp, Artificial Sequence
  Description of Artificial Sequence Synthetic oligonucleotide
ctgagtnggagncaaggcacacaggggatagg

SEQ ID NO: 138 - DNA, 32 bp, Artificial Sequence
  Description of Artificial Sequence Synthetic oligonucleotide
ctgagtgggaggcaaggcacacaggggatagg

SEQ ID NO: 139 - DNA, 33 bp, Artificial Sequence
  Description of Artificial Sequence Synthetic oligonucleotide
actgagtgggaggcaaggcacacaggggatagg

SEQ ID NO: 140 - DNA, 44 bp, Artificial Sequence
  Description of Artificial Sequence Synthetic oligonucleotide
ccatctcatccctgcgtgtcccatctgttccctccctgtctcag

SEQ ID NO: 141 - DNA, 44 bp, Artificial Sequence
  Description of Artificial Sequence Synthetic oligonucleotide
ctgagacagggagggaacagatggcaaggcacacaggggatagg

SEQ ID NO: 142 - DNA, 32 bp, Artificial Sequence; identical to SEQ ID NO: 137
  Description of Artificial Sequence Synthetic oligonucleotide

(19) United States
(12) Patent Application Publication
(10) Pub. No.: US 2011/0003701 A1
(43) Pub. Date: Jan. 6, 2011

(54) System and method for improved processing of nucleic acids for production of sequencable libraries

(75) Inventors: Gianni Calogero Ferreri, Northford, CT (US); Jan F. Simons, San Francisco, CA (US); Michael Todd Ronan, New York, NY (US); Michael Egholm, Madison, CT (US); Brian C. Godwin, North Haven, CT (US); David Roderick Riches, Hamden, CT (US); Stephen Kyle Hutchison, Branford, CT (US); Michael S. Braverman, New Haven, CT (US); Melinda D. Palmer, Hamden, CT (US); Jeffrey Jeddeloh, Verona, WI (US); Jacob Kitzman, Seattle, WA (US); Thomas J. Albert, Verona, WI (US)

Correspondence Address: Ivor R. Elrifi;Mintz, Levin, Cohn, Ferris, Glovsky and Popeo, P.C, 666 Third Avenue - 24th Floor, New York, NY, 10017, US

(73) Assignee: 454 Life Sciences Corporation

(21) Appl. No.: 12/380,139

(22) Filed: Feb. 23, 2009

Related U.S. Application Data
(60) Provisional application No. 61/031,779, filed on Feb. 27, 2008.
(60) Provisional application No. 61/032,149, filed on Feb. 28, 2008.

(51) Int. Cl. C40B 30/04; C07H 21/00; C12P 19/34; C12Q 1/68; C07H 21/04

(52) U.S. Cl. 506/9; 536/23.1; 435/91.2; 435/6

(57) ABSTRACT

An embodiment of an adaptor element for efficient target processing is described that comprises a semi-complementary double stranded nucleic acid adaptor comprising a non-complementary region and a complementary region, where the non-complementary region comprises a first amplification primer site and a second amplification primer site and the complementary region comprises a sequencing primer site and one or more inosine species.

### CROSS-REFERENCE TO RELATED APPLICATIONS

This application is related to and claims priority from U.S. Provisional Patent Application Ser. No. 61/031,779, titled “System and Method for Improved Processing of Nucleic Acids for Production of Sequencable Libraries”, filed Feb. 27, 2008; and U.S. Provisional Patent Application Ser. No. 61/032,149, titled “Methods and Systems for Multiplexed Nucleic Acid Sequence Analysis”, filed Feb. 28, 2008, each of which is hereby incorporated by reference herein in its entirety for all purposes.

### FIELD OF THE INVENTION

The present invention relates to the fields of molecular biology and nucleic acid sequencing instrumentation. More specifically, the invention relates to efficient processing of nucleic acids using methods and unique adaptor elements to produce libraries of fragments amenable for sequencing.

### BACKGROUND OF THE INVENTION

There have been a number of advancements in the field of Molecular Biology that have enabled the development of many technologies that provide great insight into the nature of biological mechanisms. The power of some of these technologies has made great impacts upon scientific discovery and hold great promise for the future. Importantly, some of these technologies are complementary to each other and may be used synergistically to speed the rate at which science gains an understanding of biological systems. It will be appreciated that the field of Molecular Biology is extremely complex and developers of such technologies may find new uses for previously known mechanisms, but the same developers will build upon new discovery and understanding of biological mechanisms derived through advances in the field of Molecular Biology.

For instance, there are a number of “nucleic acid sequencing” techniques known in the art that have delivered tremendous contributions to scientific knowledge and hold great promise for future advancements in scientific discovery as well as diagnostic application. Older nucleic acid sequencing techniques include what are referred to as Sanger type sequencing methods commonly known to those of ordinary skill in the art that employ termination and size separation techniques to identify nucleic acid composition. More recently developed sequencing techniques include classes such as what are referred to as Sequencing by Hybridization (SBH) or Sequencing by Ligation techniques. Another class of powerful sequencing techniques includes what are referred to as “sequencing-by-synthesis” techniques (SBS), and include what is referred to as the “Pyrosequencing” techniques. SBS techniques are generally employed for determining the identity or nucleic acid composition of one or more molecules in a nucleic acid sample. SBS techniques provide many desirable advantages over previously employed sequencing techniques. For example, embodiments of SBS are enabled to perform what are referred to as high throughput sequencing that generates a large volume of high quality sequence information at a low cost relative to previous techniques. A further advantage includes the simultaneous generation of sequence information from multiple template molecules in a massively parallel fashion. In other words, multiple nucleic acid molecules derived from one or more samples are simultaneously sequenced in a single process.

Typical embodiments of SBS comprise the stepwise synthesis of strands of polynucleotide molecules each complementary to a strand from a population of substantially identical template nucleic acid molecules. For example, SBS techniques typically operate by adding a single nucleotide (also referred to as a nucleotide or nucleic acid species) to each nascent polynucleotide molecule in the population where the added nucleotide species is complementary to a nucleotide species of a corresponding template molecule at a particular sequence position. The addition of the nucleic acid species to the nascent molecules typically occur in parallel for the population at the same sequence position and are detected using a variety of methods known in the art that include, but are not limited to what are referred to as pyrosequencing that detects liberated pyrophosphate molecule from incorporation events or fluorescent detection methods such as fluorescent detection techniques employing reversible or “virtual” terminators (the term virtual terminator as used herein generally refers to terminators substantially slow reaction kinetics where additional steps may be employed to stop the reaction such as the removal of reactants). Typically, the SBS process is iterative until a complete (i.e. all sequence positions of the target nucleic acid molecule are represented) or desired sequence length complementary to the template is synthesized.

In some embodiments of SBS a number of enzymatic reactions take place in order to produce a detectable signal from each incorporated nucleic acid species. In the example of the pyrosequencing SBS method referred to above what may be referred to as an enzymatic cascade is employed, where each enzyme species in the cascade operates to modify or utilize the product from a previous step. For example, as those of ordinary skill in the art understand when each nucleotide species is incorporated into the nascent strand there is a release of an inorganic pyrophosphate (also referred to as PPi) molecule into the reaction environment. The ATP sulfurylase enzyme is present in the reaction environment and converts PPi to ATP, which in turns is catalyzed by the luciferase enzyme to release a photon of light. It will also be appreciated by those of ordinary skill that additional enzymes may be used in the cascade to improve the discretion of signals between exposures to different nucleotides species as well as the overall ability to detect signals. In the present example, some embodiments may employ a number of enzymes that include one or more of, but are not limited to, apyrase that degrades unincorporated nucleotide species and ATP, exonuclease that degrades linear nucleic acid molecules, pyrophosphatase (also referred to as PPi-ase) which degrades PPi, or enzymes that inhibit activity of other enzymes. Additional examples of enzymatic improvements for signal discretion are described in U.S. patent application Ser. No 12/215,455, titled “System and Method For Adaptive Reagent Control in Nucleic Acid Sequencing”, filed Jun. 27, 2008; and Attorney Docket No 21465-538001US, titled “System and Method for Improved Signal Detection in Nucleic Acid Sequencing”, filed Jan. 29, 2009, each of which is hereby incorporated by reference herein in its entirety for all purposes.

Further, some embodiments of SBS are performed using instrumentation that automates one or more steps or operation associated with the preparation and/or sequencing methods. Some instruments employ elements such as plates with wells or other type of microreactor configuration that provide the ability to perform reactions in each of the wells or microreactors simultaneously. Additional examples of SBS techniques as well as systems and methods for massively parallel sequencing are described in U.S. Pat. Nos. 6,274,320; 6,258,568; 6,210,891; 7,211,390; 7,244,559; 7,264,929; 7,323,305; and 7,335,762 each of which is hereby incorporated by reference herein in its entirety for all purposes; and U.S. patent application Ser. No. 11/195,254, which is hereby incorporated by reference herein in its entirety for all purposes.

An additional technology that has made also made great impacts in Molecular Biology and, in some contexts may be used synergistically with nucleic acid sequencing, include the field generally referred to as “nucleic acid probe arrays” (also generally referred to as “Microarrays”). As those having skill in the art generally appreciate, Microarray technologies enable selective identification and/or enrichment of targeted nucleic acid molecules. Microarrays have been employed in many different contexts providing a wealth of information in numerous areas of biological research, as well as achieving great commercial significance. One of the principle advantages provided by

Microarray technologies is the ability to interrogate select nucleic acid molecules using targeted probes in a massively parallel manner, where some embodiments of a single Microarray may include hundreds of thousands of “probe features” each comprising hundreds of thousands of probes that target a specific nucleic acid sequence. One example of the power of Microarrays includes methods for selective “enrichment” or “complexity reduction” of populations of target nucleic acid molecules from a complex sample. The advantages of these methods include targeted selection of molecules in a massively parallel way where there may be questions as to specific characteristics of each target molecule that may include identification of the specific sequence composition of each. Thus the Microarray technology may be used synergistically with high throughput sequencing technologies to selectively enrich a population of target molecules of interest and subsequently efficiently identify the sequence composition for each. In the present example, a single Microarray can capture tens or hundreds of thousands of nucleic acid molecules from a sample by hybridization to complementary probes on the Microarray. The captured nucleic acid molecules may be subsequently eluted from the Microarray and each processed and sequenced. Also, in some embodiments of complexity reduction using probes it is not necessary to use solid phase substrates and be more broadly interpreted as “hybridization mediated” complexity reduction using solution phase probes to selectively enrich for target molecules of interest. Additional examples are described in U.S. patent application Ser. No. 11/789,135, titled “Use of microarrays for genomic representation selection”, filed Apr. 24, 2007; and Ser. No. 11/970,949 filed on Jan. 8, 2008, titled “ENRICHMENT AND SEQUENCE ANALYSIS OF GENOMIC REGIONS” each of which is hereby incorporated by reference herein in its entirety for all purposes.

It is generally desirable to continually improve technologies such as the Microarray and Sequencing technologies described above in order to enhance the abilities of scientists to provide insight into biological questions. In preferred embodiments, such improvements are aimed to reduced cost, increase throughput and efficiency, as well as to improve data quality that includes but is not limited to increased sensitivity and specificity. Therefore, it is significantly advantageous to continue to develop Microarray and nucleic acid sequencing technologies applying the knowledge and understanding of the field of Molecular Biology to provide more efficient and powerful discovery tools.

Aspects of the invention described herein employ several Molecular Biology concepts in a new and inventive way to improve the efficiency of processing samples that reduce costs, eliminate steps, and improve data quality.

### SUMMARY OF THE INVENTION

Embodiments of the invention relate to the determination of the sequence of nucleic acids. More particularly, embodiments of the invention relate to methods and systems for correcting errors in data obtained during the sequencing of nucleic acids by SBS.

An embodiment of an adaptor element for efficient target processing is described that comprises a semi-complementary double stranded nucleic acid adaptor comprising a non-complementary region and a complementary region, where the non-complementary region comprises a first amplification primer site and a second amplification primer site and the complementary region comprises a sequencing primer site and one or more inosine species. Also a kit is described that comprises the embodiment of the adaptor element.

In addition, an embodiment of a method for efficient target processing is described that comprises ligating a species of a double stranded nucleic acid adaptor to each end of a linear double stranded nucleic acid molecule to produce an adapted double stranded nucleic acid molecule, wherein the species of the double stranded nucleic acid adaptor comprises a complementary region amenable for ligation to the linear double stranded nucleic acid molecule and a non-complementary region that inhibits ligation; dissociating the adapted double stranded nucleic acid molecule to produce a first strand and a second strand each comprising a first amplification primer site and a sequencing primer site at a first end and a second amplification site at a second end; and individually amplifying the first and second strands to produce a first clonal population comprising copies of the first strand and a second clonal population comprising copies of the second strand. In some implementations the complementary region comprises one or more inosine species.

Also, an embodiment of a method for multiplex target processing and enrichment is described that comprises ligating a species of a double stranded nucleic acid adaptor to each end of a plurality of linear double stranded nucleic acid molecules from a plurality of samples to produce a pool of adapted double stranded nucleic acid molecules, wherein the species of the double stranded nucleic acid adaptor comprises a sample specific identifier element; dissociating a plurality of members from the pool adapted double stranded nucleic acid molecules to produce a first strand and a second strand from each of the dissociated members to produce a population of single stranded molecules; hybridizing a plurality of members of the population of single stranded molecules to a substrate bound capture probe, wherein the population of single stranded molecules comprises at least one member that does not hybridize to a substrate bound capture probe; eluting the hybridized members from the substrate bound capture probe to produce an enriched population of single stranded molecules; amplifying a plurality of members of the enriched population of single stranded molecules to produce a clonal population from each amplified member; individually sequencing the clonal populations to produce sequence data for each amplified member that comprises a sequence composition for the multiplex identifier element; and associating the sequence data with one of the samples using the sample specific identifier.

The above embodiments and implementations are not necessarily inclusive or exclusive of each other and may be combined in any manner that is non-conflicting and otherwise possible, whether they be presented in association with a same, or a different, embodiment or implementation. The description of one embodiment or implementation is not intended to be limiting with respect to other embodiments and/or implementations. Also, any one or more function, step, operation, or technique described elsewhere in this specification may, in alternative implementations, be combined with any one or more function, step, operation, or technique described in the summary. Thus, the above embodiment and implementations are illustrative rather than limiting.

### BRIEF DESCRIPTION OF THE DRAWINGS

The above and further features will be more clearly appreciated from the following detailed description when taken in conjunction with the accompanying drawings. In the drawings, like reference numerals indicate like structures, elements, or method steps and the leftmost digit of a reference numeral indicates the number of the figure in which the references element first appears (for example, element 130 appears first in FIG. 1). All of these conventions, however, are intended to be typical or illustrative, rather than limiting.

FIG. 1 is a functional block diagram of one embodiment of a sequencing instrument and computer system amenable for use with the described invention; and

FIG. 2A is a simplified graphical representation of one embodiment of a semi-complementary adaptor (SEQ ID NOS 140, 141 and 141, respectively, in order of appearance);

FIG. 2B is a simplified graphical representation of one embodiment of one strand of the semi-complementary adaptor of FIG. 2A that comprises a phosphate moiety on the 5′ end;

FIG. 3 is a simplified graphical representation of embodiments of the semi-complementary adaptor of FIG. 2 directionally ligated to a target nucleic acid molecule (SEQ ID NOS 140, 141, 140, and 141, respectively, in order of appearance disclosed on the left and SEQ ID NOS 140, 141, 140 and 141, respectively, in order of appearance disclosed on the right);

FIG. 4 is a simplified graphical representation of a second embodiment of a semi-complementary adaptor comprising inosine (SEQ ID NOS 135 and 142, respectively, in order of appearance); and

FIGS. 5A and 5B provide a simplified graphical representation of an embodiment of a comparison of amplification efficiencies produced using a first adaptor comprising inosine and a second adaptor lacking inosine.

### DETAILED DESCRIPTION OF THE INVENTION

As will be described in greater detail below, embodiments of the presently described invention include systems and methods for improving the processing of raw nucleic acid molecules to generate libraries of sequencable molecules.

### a. General

The term “flowgram” or “pyrogram” may be used interchangeably herein and generally refer to a graphical representation of sequence data generated by SBS methods.

The term “read” or “sequence read” as used herein generally refers to the entire sequence data obtained from a single nucleic acid template molecule or a population of a plurality of substantially identical copies of the template nucleic acid molecule.

The terms “run” or “sequencing run” as used herein generally refer to a series of sequencing reactions performed in a sequencing operation of one or more template nucleic acid molecules.

The term “flow” as used herein generally refers to a serial or iterative cycle of addition of solution to an environment comprising a template nucleic acid molecule, where the solution may include a nucleotide species for addition to a nascent molecule or other reagent such as buffers or enzymes that may be employed in a sequencing reaction or to reduce carryover or noise effects from previous flow cycles of nucleotide species.

The term “flow cycle” as used herein generally refers to a sequential series of flows where a nucleotide species is flowed once during the cycle (i.e. a flow cycle may include a sequential addition in the order of T, A, C, G nucleotide species, although other sequence combinations are also considered part of the definition). Typically the flow cycle is a repeating cycle having the same sequence of flows from cycle to cycle.

The term “read length” as used herein generally refers to an upper limit of the length of a template molecule that may be reliably sequenced. There are numerous factors that contribute to the read length of a system and/or process including, but not limited to the degree of GC content in a template nucleic acid molecule.

The term “test fragment”, or “TF” as used herein generally refers to a nucleic acid element of known sequence composition that may be employed for quality control, calibration, or other related purposes.

A “nascent molecule” generally refers to a DNA strand which is being extended by the template-dependent DNA polymerase by incorporation of nucleotide species which are complementary to the corresponding nucleotide species in the template molecule.

The terms “template nucleic acid”, “template molecule”, “target nucleic acid”, or “target molecule” generally refer to a nucleic acid molecule that is the subject of a sequencing reaction from which sequence data or information is generated.

The term “nucleotide species” as used herein generally refers to the identity of a nucleic acid monomer including purines (Adenine, Guanine) and pyrimidines (Cytosine, Uracil, Thymine) typically incorporated into a nascent nucleic acid molecule.

The term “monomer repeat” or “homopolymers” as used herein generally refers to two or more sequence positions comprising the same nucleotide species (i.e. a repeated nucleotide species).

The term “homogeneous extension”, as used herein, generally refers to the relationship or phase of an extension reaction where each member of a population of substantially identical template molecules is homogenously performing the same extension step in the reaction.

The term “completion efficiency” as used herein generally refers to the percentage of nascent molecules that are properly extended during a given flow.

The term “incomplete extension rate” as used herein generally refers to the ratio of the number of nascent molecules that fail to be properly extended over the number of all nascent molecules.

The term “genomic library” or “shotgun library” as used herein generally refers to a collection of molecules derived from and/or representing an entire genome (i.e. all regions of a genome) of an organism or individual.

The term “amplicon” as used herein generally refers to selected amplification products such as those produced from Polymerase Chain Reaction or Ligase Chain Reaction techniques.

The term “key sequence” or “key element” as used herein generally refers to a nucleic acid sequence element (typically of about 4 sequence positions, i.e. TGAC or other combination of nucleotide species) associated with a template nucleic acid molecule in a known location (i.e. typically included in a ligated adaptor element) comprising known sequence composition that is employed as a quality control reference for sequence data generated from template molecules. The sequence data passes the quality control if it includes the known sequence composition associated with a Key element in the correct location.

The term “keypass” or “keypass well” as used herein generally refers to the sequencing of a full length nucleic acid test sequence of known sequence composition (also referred to as a “test fragment”) in a reaction well, where the accuracy of the sequence derived from keypass test sequence is compared to the known sequence composition and used to measure of the accuracy of the sequencing and for quality control. In typical embodiments a proportion of the total number of wells in a sequencing run will be keypass wells which may in some embodiments be regionally distributed or specific.

The term “blunt end” or “blunt ended” as used herein generally refers to a linear double stranded nucleic acid molecule having an end that terminates with a pair of complementary nucleotide base species, where a pair of blunt ends are always compatible for ligation to each other.

The term “sticky end” or “overhang” as used herein is generally interpreted consistently with the understanding of one of ordinary skill in the related art and includes a linear double stranded nucleic acid molecule having one or more unpaired nucleotide species at the end of one strand of the molecule, where the unpaired nucleotide species may exist on either strand and include a single base position or a plurality of base positions (also sometimes referred to as “cohesive end”). The term “bead” or “bead substrate” as used herein generally refers to a any type of bead of any convenient size and fabricated from any number of known materials such as cellulose, cellulose derivatives, acrylic resins, glass, silica gels, polystyrene, gelatin, polyvinyl pyrrolidone, co-polymers of vinyl and acrylamide, polystyrene cross-linked with divinylbenzene or the like (as described, e.g., in Merrifield, Biochemistry 1964, 3, 1385-1390), polyacrylamides, latex gels, polystyrene, dextran, rubber, silicon, plastics, nitrocellulose, natural sponges, silica gels, control pore glass, metals, cross-linked dextrans (e.g., Sephadex™) agarose gel (Sepharose™), and other solid phase bead supports known to those of skill in the art.

Some exemplary embodiments of systems and methods associated with sample preparation and processing, generation of sequence data, and analysis of sequence data are generally described below, some or all of which are amenable for use with embodiments of the presently described invention. In particular the exemplary embodiments of systems and methods for preparation of template nucleic acid molecules, amplification of template molecules, generating target specific amplicons and/or genomic libraries, sequencing methods and instrumentation, and computer systems are described.

In typical embodiments, the nucleic acid molecules derived from an experimental or diagnostic sample must be prepared and processed from its raw form into template molecules amenable for high throughput sequencing. The processing methods may vary from application to application resulting in template molecules comprising various characteristics. For example, in some embodiments of high throughput sequencing it is preferable to generate template molecules with a sequence or read length that is at least the length a particular sequencing method can accurately produce sequence data for. In the present example, the length may include a range of about 25-30 base pairs, about 50-100 base pairs, about 200-300 base pairs, about 350-500 base pairs, greater than 500 base pairs, or other length amenable for a particular sequencing application. In some embodiments, nucleic acids from a sample, such as a genomic sample, are fragmented using a number of methods known to those of ordinary skill in the art. In preferred embodiments, methods that randomly fragment (i.e. do not select for specific sequences or regions) nucleic acids and may include what is referred to as nebulization or sonication methods. It will however, be appreciated that other methods of fragmentation such as digestion using restriction endonucleases may be employed for fragmentation purposes. Also in the present example, some processing methods may employ size selection methods known in the art to selectively isolate nucleic acid fragments of the desired length.

Also, it is preferable in some embodiments to associate additional functional elements with each template nucleic acid molecule. The elements may be employed for a variety of functions including, but not limited to, primer sequences for amplification and/or sequencing methods, quality control elements, unique identifiers (also referred to as multiplex identifiers) that encode various associations such as with a sample of origin or patient, or other functional element. For example, some embodiments may associate priming sequence elements or regions comprising complementary sequence composition to primer sequences employed for amplification and/or sequencing. Further, the same elements may be employed for what may be referred to as “strand selection” and immobilization of nucleic acid molecules to a solid phase substrate. In the present example, two sets of priming sequence regions (hereafter referred to as priming sequence A, and priming sequence B) may be employed for strand selection where only single strands having one copy of priming sequence A and one copy of priming sequence B is selected and included as the prepared sample. The same priming sequence regions may be employed in methods for amplification and immobilization where, for instance priming sequence B may be immobilized upon a solid substrate and amplified products are extended therefrom.

Additional examples of sample processing for fragmentation, strand selection, and addition of functional elements and adaptors are described in U.S. patent application Ser. No. 10/767,894, titled “Method for preparing single-stranded DNA libraries”, filed Jan. 28, 2004; and U.S. patent application Ser. No. 12/156,242, titled “System and Method for Identification of Individual Samples from a Multiplex Mixture”, filed May 29, 2008, each of which is hereby incorporated by reference herein in its entirety for all purposes.

Various examples of systems and methods for performing amplification of template nucleic acid molecules to generate populations of substantially identical copies are described. It will be apparent to those of ordinary skill that it is desirable in some embodiments of SBS to generate many copies of each nucleic acid element to generate a stronger signal when one or more nucleotide species is incorporated into each nascent molecule associated with a copy of the template molecule. There are many techniques known in the art for generating copies of nucleic acid molecules such as, for instance, amplification using what are referred to as bacterial vectors, “Rolling Circle” amplification (described in U.S. Pat. Nos. 6,274,320 and 7,211,390, incorporated by reference above) and Polymerase Chain Reaction (PCR) methods, each of the techniques are applicable for use with the presently described invention. One PCR technique that is particularly amenable to high throughput applications include what are referred to as emulsion PCR methods (also referred to as emPCR™ methods).

Typical embodiments of emulsion PCR methods include creating a stable emulsion of two immiscible substances creating aqueous droplets within which reactions may occur. In particular, the aqueous droplets of an emulsion amenable for use in PCR methods may include a first fluid such as a water based fluid suspended or dispersed in what may be referred to as a discontinuous phase within another fluid in what may be referred to as a continuous phase such as an oil based fluid. Further, some emulsion embodiments may employ surfactants that act to stabilize the emulsion that may be particularly useful for specific processing methods such as PCR. Some embodiments of surfactant may include non-ionic surfactants such as sorbitan monooleate (also referred to as Span™ 80), polyoxyethylenesorbitan monooleate (also referred to as Tween™ 80), or in some preferred embodiments dimethicone copolyol (also referred to as Abil® EM90), polysiloxane, polyalkyl polyether copolymer, polyglycerol esters, poloxamers, and PVP/hexadecane copolymers (also referred to as Unimer U-151), or in more preferred embodiments a high molecular weight silicone polyether in cyclopentasiloxane (also referred to as DC 5225C available from Dow Corning).

The droplets of an emulsion may also be referred to as compartments, microcapsules, microreactors, microenvironments, or other name commonly used in the related art. The aqueous droplets may range in size depending on the composition of the emulsion components or composition, contents contained therein, and formation technique employed. The described emulsions create the microenvironments within which chemical reactions, such as PCR, may be performed. For example, template nucleic acids and all reagents necessary to perform a desired PCR reaction may be encapsulated and chemically isolated in the droplets of an emulsion. Additional surfactants or other stabilizing agent may be employed in some embodiments to promote additional stability of the droplets as described above. Thermocycling operations typical of PCR methods may be executed using the droplets to amplify an encapsulated nucleic acid template resulting in the generation of a population comprising many substantially identical copies of the template nucleic acid. In some embodiments, the population within the droplet may be referred to as a “clonally isolated”, “compartmentalized”, “sequestered”, “encapsulated”, or “localized” population. Also in the present example, some or all of the described droplets may further encapsulate a solid substrate such as a bead for attachment of template or other type of nucleic acids, reagents, labels, or other molecules of interest.

Embodiments of an emulsion useful with the presently described invention may include a very high density of droplets or microcapsules enabling the described chemical reactions to be performed in a massively parallel way. Additional examples of emulsions employed for amplification and their uses for sequencing applications are described in U.S. patent application Ser. Nos. 10/861,930; 10/866,392; 10/767,899; 11/045,678 each of which are hereby incorporated by reference herein in its entirety for all purposes.

Also, embodiments that generate target specific amplicons for sequencing may be employed with the presently described invention that include using sets of specific nucleic acid primers to amplify a selected target region or regions from a sample comprising the target nucleic acid. Further, the sample may include a population of nucleic acid molecules that are known or suspected to contain sequence variants and the primers may be employed to amplify and provide insight into the distribution of sequence variants in the sample. For example a method for identifying a sequence variant by specific amplification and sequencing of multiple alleles in a nucleic acid sample may be performed. The nucleic acid is first subjected to amplification by a pair of PCR primers designed to amplify a region surrounding the region of interest or segment common to the nucleic acid population. Each of the products of the PCR reaction (amplicons) is subsequently further amplified individually in separate reaction vessels such as an emulsion based vessel described above. The resulting amplicons (referred to herein as second amplicons), each derived from one member of the first population of amplicons, are sequenced and the collection of sequences, from different emulsion PCR amplicons, are used to determine an allelic frequency.

Some advantages of the described target specific amplification and sequencing methods include a higher level of sensitivity than previously achieved. Further, embodiments that employ high throughput sequencing instrumentation such as for instance embodiments that employ what is referred to as a PicoTiterPlate® array (also sometimes referred to as a PTP® plate or array) of wells provided by 454 Life Sciences Corporation, the described methods can be employed to sequence over 100,000 or over 300,000 different copies of an allele per run or experiment. Also, the described methods provide a sensitivity of detection of low abundance alleles which may represent 1% or less of the allelic variants. Another advantage of the methods includes generating data comprising the sequence of the analyzed region. Importantly, it is not necessary to have prior knowledge of the sequence of the locus being analyzed.

Additional examples of target specific amplicons for sequencing are described in U.S. patent application Ser. No. 11/104,781, titled “Methods for determining sequence variants using ultra-deep sequencing”, filed Apr. 12, 2005; and PCT Patent Application Serial No. US 2008/003424, titled “System and Method for Detection of HIV Drug Resistant Variants”, filed Mar. 14, 2008, each of which is hereby incorporated by reference herein in its entirety for all purposes.

Further, embodiments of sequencing may include Sanger type techniques, techniques generally referred to as Sequencing by Hybridization (SBH) or Sequencing by Incorporation (SBI) that may include what is referred to as polony sequencing techniques; nanopore, waveguide and other single molecule detection techniques; or reversible terminator techniques. As described above a preferred technique may include Sequencing by Synthesis methods. For example, some SBS embodiments sequence populations of substantially identical copies of a nucleic acid template and typically employ one or more oligonucleotide primers designed to anneal to a predetermined, complementary position of the sample template molecule or one or more adaptors attached to the template molecule. The primer/template complex is presented with a nucleotide species in the presence of a nucleic acid polymerase enzyme. If the nucleotide species is complementary to the nucleic acid species corresponding to a sequence position on the sample template molecule that is directly adjacent to the 3′ end of the oligonucleotide primer, then the polymerase will extend the primer with the nucleotide species. Alternatively, in some embodiments the primer/template complex is presented with a plurality of nucleotide species of interest (typically A, G, C, and T) at once, and the nucleotide species that is complementary at the corresponding sequence position on the sample template molecule directly adjacent to the 3′ end of the oligonucleotide primer is incorporated. In either of the described embodiments, the nucleotide species may be chemically blocked (such as at the 3′-O position) to prevent further extension, and need to be deblocked prior to the next round of synthesis. It will also be appreciated that the process of adding a nucleotide species to the end of a nascent molecule is substantially the same as that described above for addition to the end of a primer.

As described above, incorporation of the nucleotide species can be detected by a variety of methods known in the art, e.g. by detecting the release of pyrophosphate (PPi) (examples described in U.S. Pat. Nos. 6,210,891; 6,258,568; and 6,828,100, each of which is hereby incorporated by reference herein in its entirety for all purposes), or via detectable labels bound to the nucleotides. Some examples of detectable labels include but are not limited to mass tags and fluorescent or chemiluminescent labels. In typical embodiments, unincorporated nucleotides are removed, for example by washing. Further, in some embodiments the unincorporated nucleotides may be subjected to enzymatic degradation such as, for instance, degradation using the apyrase or pyrophosphatase enzymes as described in U.S. patent application Ser. No. 12/215,455, titled “System and Method for Adaptive Reagent Control in Nucleic Acid Sequencing”, filed Jun. 27, 2008; and Attorney Docket No 21465-538001 US, titled “System and Method for Improved Signal Detection in Nucleic Acid Sequencing”, filed Jan. 29, 2009; each of which is hereby incorporated by reference herein in its entirety for all purposes.

In the embodiments where detectable labels are used, they will typically have to be inactivated (e.g. by chemical cleavage or photobleaching) prior to the following cycle of synthesis. The next sequence position in the template/polymerase complex can then be queried with another nucleotide species, or a plurality of nucleotide species of interest, as described above. Repeated cycles of nucleotide addition, extension, signal acquisition, and washing result in a determination of the nucleotide sequence of the template strand. Continuing with the present example, a large number or population of substantially identical template molecules (e.g. 10³, 10⁴, 10⁵, 10⁶ or 10⁷ molecules) are typically analyzed simultaneously in any one sequencing reaction, in order to achieve a signal which is strong enough for reliable detection.

In addition, it may be advantageous in some embodiments to improve the read length capabilities and qualities of a sequencing process by employing what may be referred to as a “paired-end” sequencing strategy. For example, some embodiments of sequencing method have limitations on the total length of molecule from which a high quality and reliable read may be generated. In other words, the total number of sequence positions for a reliable read length may not exceed 25, 50, 100, or 150 bases depending on the sequencing embodiment employed. A paired-end sequencing strategy extends reliable read length by separately sequencing each end of a molecule (sometimes referred to as a “tag” end) that comprise a fragment of an original template nucleic acid molecule at each end joined in the center by a linker sequence. The original positional relationship of the template fragments is known and thus the data from the sequence reads may be re-combined into a single read having a longer high quality read length. Further examples of paired-end sequencing embodiments are described in U.S. patent application Ser. No. 11/448,462, titled “Paired end sequencing”, filed Jun. 6, 2006, and in Attorney Docket No. 21465-537001 US, titled “Paired end sequencing”, filed Jan. 28, 2009, each of which is hereby incorporated by reference herein in its entirety for all purposes.

Some examples of SBS apparatus may implement some or all of the methods described above and may include one or more of a detection device such as a charge coupled device (i.e. CCD camera) or a confocal type architecture, a microfluidics chamber or flow cell, a reaction substrate, and/or a pump and flow valves. Taking the example of pyrophosphate based sequencing, embodiments of an apparatus may employ a chemiluminescent detection strategy that produces an inherently low level of background noise.

In some embodiments, the reaction substrate for sequencing may include what is referred to as a PTP® array, as described above, formed from a fiber optics faceplate that is acid-etched to yield hundreds of thousands or more of very small wells each enabled to hold a population of substantially identical template molecules (i.e. some preferred embodiments comprise about 3.3 million wells on a 70×75 mm PTP® array at a 35 μm well to well pitch). In some embodiments, each population of substantially identical template molecule may be disposed upon a solid substrate such as a bead, each of which may be disposed in one of said wells. For example, an apparatus may include a reagent delivery element for providing fluid reagents to the PTP plate holders, as well as a CCD type detection device enabled to collect photons of light emitted from each well on the PTP plate. An example of reaction substrates comprising characteristics for improved signal recognition is described in U.S. patent application Ser. No 11/215,458, titled “THIN-FILM COATED MICROWELL ARRAYS AND METHODS OF MAKING SAME”, filed Aug. 30, 2005, which is hereby incorporated by reference herein in its entirety for all purposes. Further examples of apparatus and methods for performing SBS type sequencing and pyrophosphate sequencing are described in U.S. Pat. No 7,323,305 and U.S. patent application Ser. No. 11/195,254 both of which are incorporated by reference above.

In addition, systems and methods may be employed that automate one or more sample preparation processes, such as the emPCR™ process described above. For example, automated systems may be employed to provide an efficient solution for generating an emulsion for emPCR processing, performing PCR Thermocycling operations, and enriching for successfully prepared populations of nucleic acid molecules for sequencing. Examples of automated sample preparation systems are described in U.S. patent application Ser. No. 11/045,678, titled “Nucleic acid amplification with continuous flow emulsion”, filed Jan. 28, 2005, which is hereby incorporated by reference herein in its entirety for all purposes.

Also, the systems and methods of the presently described embodiments of the invention may include implementation of some design, analysis, or other operation using a computer readable medium stored for execution on a computer system. For example, several embodiments are described in detail below to process detected signals and/or analyze data generated using SBS systems and methods where the processing and analysis embodiments are implementable on computer systems.

An exemplary embodiment of a computer system for use with the presently described invention may include any type of computer platform such as a workstation, a personal computer, a server, or any other present or future computer. Computers typically include known components such as a processor, an operating system, system memory, memory storage devices, input-output controllers, input-output devices, and display devices. It will be understood by those of ordinary skill in the relevant art that there are many possible configurations and components of a computer and may also include cache memory, a data backup unit, and many other devices.

Display devices may include display devices that provide visual information, this information typically may be logically and/or physically organized as an array of pixels. An interface controller may also be included that may comprise any of a variety of known or future software programs for providing input and output interfaces. For example, interfaces may include what are generally referred to as “Graphical User Interfaces” (often referred to as GUI's) that provides one or more graphical representations to a user. Interfaces are typically enabled to accept user inputs using means of selection or input known to those of ordinary skill in the related art.

In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft Windows Powershell that employs object-oriented type programming architectures such as the Microsoft .NET framework.

Those of ordinary skill in the related art will appreciate that interfaces may include one or more GUI's, CLI's or a combination thereof.

A processor may include a commercially available processor such as a Centrino®, Core™ 2, Itanium® or Pentium® processor made by Intel Corporation, a SPARC® processor made by Sun Microsystems, an Athalon™ or Opteron™ processor made by AMD corporation, or it may be one of other processors that are or will become available. Some embodiments of a processor may include what is referred to as Multi-core processor and/or be enabled to employ parallel processing technology in a single or multi-core configuration. For example, a multi-core architecture typically comprises two or more processor “execution cores”. In the present example each execution core may perform as an independent processor that enables parallel execution of multiple threads. In addition, those of ordinary skill in the related will appreciate that a processor may be configured in what is generally referred to as 32 or 64 bit architectures, or other architectural configurations now known or that may be developed in the future.

A processor typically executes an operating system, which may be, for example, a Windows®-type operating system (such as Windows® XP or Windows Vista®) from the Microsoft Corporation; the Mac OS X operating system from Apple Computer Corp. (such as Mac OS X v10.5 “Leopard” or “Snow Leopard” operating systems); a Unix® or Linux-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

System memory may include any of a variety of known or future memory storage devices. Examples include any commonly available random access memory (RAM), magnetic medium such as a resident hard disk or tape, an optical medium such as a read and write compact disc, or other memory storage device. Memory storage devices may include any of a variety of known or future devices, including a compact disk drive, a tape drive, a removable hard disk drive, USB or flash drive, or a diskette drive. Such types of memory storage devices typically read from, and/or write to, a program storage medium (not shown) such as, respectively, a compact disk, magnetic tape, removable hard disk, USB or flash drive, or floppy diskette. Any of these program storage media, or others now in use or that may later be developed, may be considered a computer program product. As will be appreciated, these program storage media typically store a computer software program and/or data. Computer software programs, also called computer control logic, typically are stored in system memory and/or the program storage device used in conjunction with memory storage device.

In some embodiments, a computer program product is described comprising a computer usable medium having control logic (computer software program, including program code) stored therein. The control logic, when executed by a processor, causes the processor to perform functions described herein. In other embodiments, some functions are implemented primarily in hardware using, for example, a hardware state machine. Implementation of the hardware state machine so as to perform the functions described herein will be apparent to those skilled in the relevant arts.

Input-output controllers could include any of a variety of known devices for accepting and processing information from a user, whether a human or a machine, whether local or remote. Such devices include, for example, modem cards, wireless cards, network interface cards, sound cards, or other types of controllers for any of a variety of known input devices. Output controllers could include controllers for any of a variety of known display devices for presenting information to a user, whether a human or a machine, whether local or remote. In the presently described embodiment, the functional elements of a computer communicate with each other via a system bus. Some embodiments of a computer may communicate with some functional elements using network or other types of remote communications.

As will be evident to those skilled in the relevant art, an instrument control and/or a data processing application, if implemented in software, may be loaded into and executed from system memory and/or a memory storage device. All or portions of the instrument control and/or data processing applications may also reside in a read-only memory or similar device of the memory storage device, such devices not requiring that the instrument control and/or data processing applications first be loaded through input-output controllers. It will be understood by those skilled in the relevant art that the instrument control and/or data processing applications, or portions of it, may be loaded by a processor in a known manner into system memory, or cache memory, or both, as advantageous for execution.

Also a computer may include one or more library files, experiment data files, and an internet client stored in system memory. For example, experiment data could include data related to one or more experiments or assays such as detected signal values, or other values associated with one or more SBS experiments or processes. Additionally, an internet client may include an application enabled to accesses a remote service on another computer using a network and may for instance comprise what are generally referred to as “Web Browsers”. In the present example some commonly employed web browsers include Microsoft® Internet Explorer 7 available from Microsoft Corporation, Mozilla Firefox® 2 from the Mozilla Corporation, Safari 1.2 from Apple Computer Corp., or other type of web browser currently known in the art or to be developed in the future. Also, in the same or other embodiments an internet client may include, or could be an element of, specialized software applications enabled to access remote information via a network such as a data processing application for SBS applications.

A network may include one or more of the many various types of networks well known to those of ordinary skill in the art. For example, a network may include a local or wide area network that employs what is commonly referred to as a TCP/IP protocol suite to communicate. A network may include a network comprising a worldwide system of interconnected computer networks that is commonly referred to as the internet, or could also include various intranet architectures. Those of ordinary skill in the related arts will also appreciate that some users in networked environments may prefer to employ what are generally referred to as “firewalls” (also sometimes referred to as Packet Filters, or Border Protection Devices) to control information traffic to and from hardware and/or software systems. For example, firewalls may comprise hardware or software elements or some combination thereof and are typically designed to enforce security policies put in place by users, such as for instance network administrators, etc.

### b. Embodiments of the Presently Described Invention

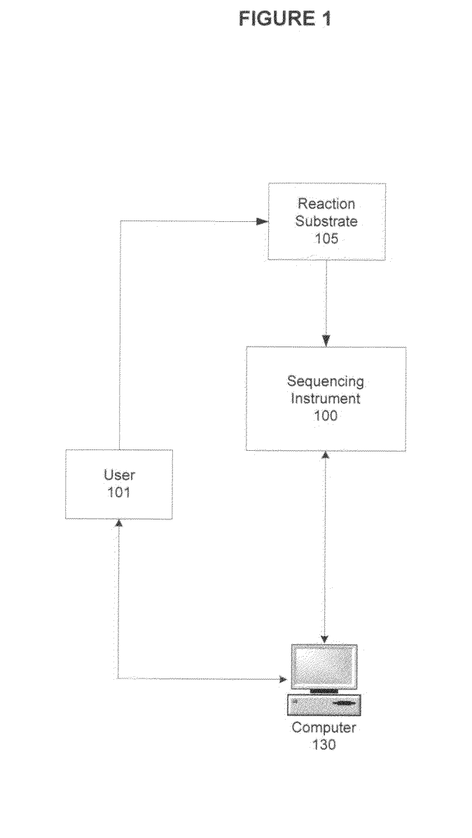

As described above, the described inventions comprise systems and methods for efficient processing of nucleic acids to produce sequencable libraries of template molecules. In the described embodiments, one or more instrument elements are employed that automate one or more process steps for introducing reactants, including enzymes, as well as for the steps of measuring and adjusting. For example, embodiments of a sequencing method may be executed using instrumentation and control software to automate and carry out some or all process steps. FIG. 1 provides an illustrative example of sequencing instrument 100 that comprises optic and fluidic subsystems. Embodiments of sequencing instrument 100 employed to execute sequencing processes may include various fluidic components in fluidic subsystem, various optical components in optic subsystem, and one or more computer components such as computer 130 that may for instance execute system software or firmware that provides instructional control of one or more of the components. In the present example, sequencing instrument 100 and/or computer 130 may include some or all of the components and characteristics of the embodiments generally described above.

Embodiments of the invention include a unique adaptor element that is associated with a target nucleic acid. The adapted target nucleic acid is subsequently processed using various methods where the characteristics of the adaptor provide a substantial increase in processing efficiency over previously employed adaptor embodiments. As will be explained in greater detail below, there are a number efficiency improvements attributable to the adaptor characteristics, such as a reduction in the number of processing steps necessary to achieve a similar result as previous adaptor embodiments (i.e. the production of a library of single stranded template molecules). Further efficiency improvements also include a reduction or elimination of components and/or reagents required for processing by previously employed adaptor embodiments.

In preferred embodiments the adaptor of the invention comprises several component elements that confer desirable characteristics to the adaptor that are particularly advantageous for use in particular processing steps. The advantages conferred by these component elements enable substantial improvements over processing target molecules operatively coupled to previous adaptor embodiments. For example, processing methods using previous adaptor embodiments are described in U.S. patent application Ser. No. 10/767,894, incorporated by reference above that employs two distinct adaptor species (referred to as Adaptor A and Adaptor B) that are randomly ligated to the ends each target nucleic acid molecule. In the present example, the individual characteristics of the A and B adaptor species make it necessary that each adapted target molecule employed in a sequencing reaction include both an A and B adaptor (i.e. one of each species ligated to an end of the target, represented as A/B adaptor combination), and thus do to the random nature of the ligation step (i.e. produces A/A and B/B adapted molecules) subsequent processing steps must be taken to insure that only molecules with an A/B adaptor combination are selected.

The invention provides a substantial improvement over processing with the combination of A/B adaptor species because there is only a single adaptor species that performs the same functions as the A/B adaptor species combination as well as additional advantages that will be illustrated further below. One important characteristic possessed by the adaptor of the invention is that it has what will be referred to herein as “directional” characteristics and strand specific elements that enable the adaptor to ligate to each end of a linear target nucleic acid molecule in a desired orientation. For example, the directional characteristic of the adaptor species of the invention is derived, at least in part, on the directional nature and base pairing relationship of the individual strands of the molecule. The proper orientation of the adaptor at each end of the target molecule appropriately positions the specific elements of each strand of the adaptor for optimal use in subsequent process steps such as, for instance, amplification and/or sequencing steps.

Another advantage of the adaptor embodiments of the invention over the previously described A/B adaptor embodiments includes the use of both strands of the adapted target molecule in subsequent steps as opposed to the production of only a single useable strand from each double stranded adapted target molecule. For example, the single adaptor species of the presently described invention eliminates the need for strand selection steps required by the A/B adaptor embodiments and produces two sequencable templates from each adapted double stranded molecule.

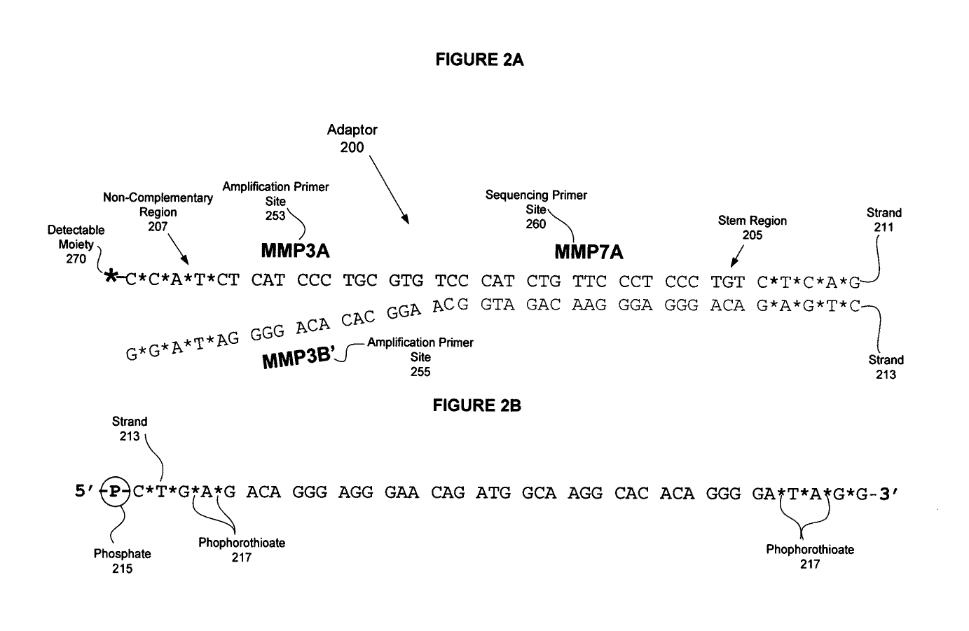

FIG. 2A provides an illustrative example of one embodiment of adaptor 200, sometimes referred to as a “Y-Adaptor” and is a “semi-complementary” double stranded nucleic acid molecule comprising stem region 205 and non-complementary region 207. The term “semi-complementary” as used herein generally refers to the complementary nature of nucleotide species at sequence positions in the molecule, where a first region comprises a sequence composition between strands that is complementary and a second region that comprises a non-complementary sequence composition (sometimes also referred to as a “frayed end”). Those of ordinary skill in the related art will appreciate that individual strands of stem region 205 and non-complementary region 207 follow the Watson-Crick base pairing rules based upon the sequence composition of each strand. It will be additionally appreciated that there may be some degree of complementarity at some sequence positions in non-complementary region 207 which are negligible as long as the strands within region 207 do not anneal. However, reducing the number of sequence positions having complementarity as much as possible is desirable. For example, embodiments of adaptor 200 include strand 211 and strand 213 where the nucleotide composition at each sequence position between strands 211 and 213 in stem region 205 is complementary and bind forming a double stranded region. Further, the nucleotide composition between strands 211 and 213 in non-complementary region 207 is non-complementary and do not bind remaining substantially independent single strands (may also be referred to as “arms”). In the present example, the sequence length of stem region 205 may vary depending on the embodiment and for instance may include a length of 12, 15, 24 or more sequence positions (also referred to as base positions). Similarly, the sequence length of non-complementary region 207 may vary depending on the embodiment. The length of region 205 or 207 may in some cases be dependent upon one or more sequence elements or components encompassed within such as primer sequences, quality control elements, unique identifier elements, or other sequence element known in the art, or some combination thereof.

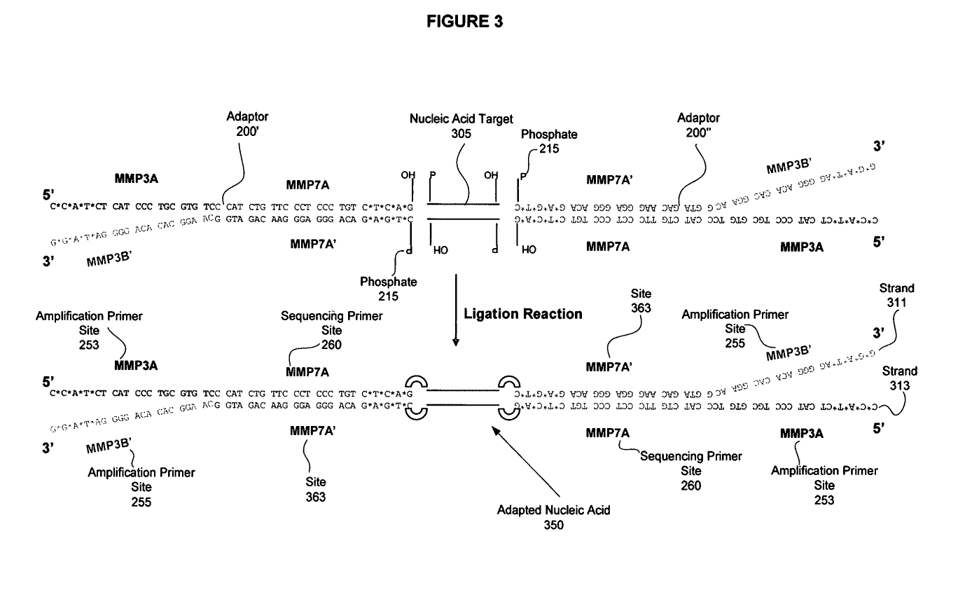

Also illustrated in FIG. 2A are several functional components positionally located in adaptor 200 to provide functionality when directionally ligated to a target nucleic acid molecule. For example, amplification primer sites 253 and 255 are positioned in non-complementary region 207 on strands 211 and 213 respectively. Sites 253 and 255 are generally employed in a PCR type amplification reaction when located on the same strand, where the nucleic acid sequence composition located between the primer sites is amplified. Another functional element of some embodiments of adaptor 200 include sequencing primer site 260 that, as described above, may provide a primer site for certain sequencing methods. The importance of the positional location of sites 253, 255, and will be discussed in greater detail below with respect to FIG. 3.

FIG. 2B provides an illustrative example of strand 213 comprising phosphate 215 on the 5′ end. For example, phosphate 215 may include a phosphate moiety that contributes to the directionality of adaptor 200 where the phosphate promotes ligation of adaptor 200 to the ends of a target molecule. Those of ordinary skill in the related art will appreciate that phosphate 215 is associated with the 5′ end of strand 213 which is beneficial for ligation of the 5′ end of adaptor 200 to the 3′ end of a target nucleic acid molecule. In the example presented in FIG. 2A, stem region 205 is “blunt ended” and ligatable with blunt ended target molecules irrespective of the base composition of either the end of stem region 205 or the end of target nucleic acid 305 illustrated in FIG. 3. However in some embodiments it may be advantageous to employ what is referred to as an “overhang” or “sticky end” of stem region 205 for ligation to an end of target nucleic acid 305 comprising a complementary sticky end as will be described in greater detail below with respect to FIG. 3.

Also illustrated in FIG. 2B is phosphorothioate 217 that represents phosphorothioate nucleotide species in the sequence composition. Those of ordinary skill in the related art will appreciate that “phosphorothioates” are analogues of nucleotide species that comprise a sulfur molecule in place of an oxygen molecule as one of the non-bridging ligands bonded to phosphorus. In embodiments of adaptor 200 or 400, the incorporation of one or more embodiments of phosphorothioate 217 into the sequence composition confers resistance to exonuclease digestion as well as providing improvement to ligation efficiency.

FIG. 3 provides an illustrative example of two embodiments of adaptor 200, illustrated as adaptor 200′ and adaptor 200″, associated for directional ligation to each end of target nucleic acid 305. General description of preparing nucleic acid target molecules that includes methods for fragmentation, blunt end polishing, ligation methods (including associated methods such as “nick fill-in” reactions), and other related processing steps are described in U.S. patent application Ser. No. 10/767,894, incorporated by reference above. Those of ordinary skill in the related art will appreciate that nucleic acid target 305 may typically comprise an unknown sequence composition and may be “phosphorylated” at the 5′ ends of individual stands as illustrated in FIG. 3 for ligation efficiency. In the example illustrated in FIG. 3, the blunt end of adaptors 200′ and 200″ align to the blunted ends of target nucleic acid 305 where 5′ phosphate 215 aligns with a 3′ OH group associated with the ends of the strands of target 305 and are ligated so that the adaptors 200′ and 200″ are in an “inverted” relationship relative to each other forming adapted nucleic acid 360. It will also be appreciated by those of ordinary skill that the structure of non-complementary region 207 inhibits ligation of the end of region 207 to the double stranded end of a target fragment. For instance, it is generally appreciated that non-complementary strands of double stranded nucleic acid molecules interfere with the ability of a ligase enzyme to join another nucleic acid to the non-complementary end. Using the example of adaptor 200, both stands 211 and 213 in stem region 205 are complementary so that a ligase enzyme preferentially joins stem region 205 to another nucleic acid over non-complementary region 207. Thus, the structural characteristics of each end of adaptor 200 and position of phosphate 215 provide directionality to adaptor 200 with respect to ligation with the ends of target nucleic acid molecules.

Further, as described above, it may be advantageous in some embodiments to employ “sticky ends” for ligation of adaptor 200 to target molecule 305. Some of the advantages of using sticky end ligation include further promoting the directional nature of the adaptor/target ligation, inhibition of target concatemer formation, inhibition of adaptor dimer formation, and inhibition of the circularization of target molecules. In some embodiments, an overhang comprising a single base position on the end of each nucleic acid molecule to be joined is sufficient for providing the various advantages listed above, however it will be appreciated that longer overhangs may also be employed. In the same or alternative embodiments the overhangs may be reliably created using methods known in the art. One embodiment may include a single base overhang where an A nucleotide species is employed as an overhang on one nucleic acid molecule and a T nucleotide species is employed as an overhang on a second nucleic acid molecule.

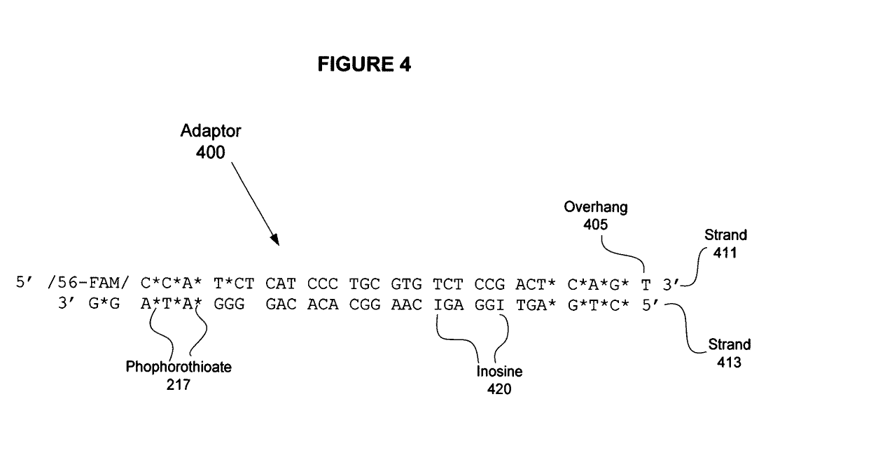

For example, FIG. 4 provides an illustrative representation of adaptor 400 may synthesized with a T overhang on strand 411 (at the 3′ associated with stem region 205). Nucleic acid target 305 may be fragmented using any of the methods known in the art and as described in U.S. patent application Ser. No. 10/767,894 incorporated by reference above, and the ends of the nucleic acid fragments may be polished to remove overhangs where the sequence composition may be unknown. Next the addition of a single base overhang comprising an A nucleotide species to the strands with 3′ ends of the fragments is performed using various methods. A first method uses the “extendase” properties of Taq polymerase. In the present example, the A extension may be achieved within the end polishing reaction buffer that includes T4 Polymerase and T4 Polynucleotide Kinase (hereafter referred to as PNK) at a temperature of 25° C. for 20 minutes to the T4 polymerase and PNK activity. Next the temperature is set to 72° C. for 20 minutes for the incorporation of the A nucleotide species and inactivation of the T4 polymerase and PNK. The reactions may also be cleaned up using SPRI technology or purification columns.

Also, some embodiments of adaptor 200 or 400 may include a detectable moiety that enables direct quantification of the number of nucleic acid molecules in a volume rather than employing quantification methods such as measurements of total mass of nucleic acid molecules and an estimation of the average size of the molecules. In some preferred embodiments the detectable moiety may include a fluorescent moiety that allows for easy, efficient, and accurate quantitation of molecule numbers via detection of light emitted from the attached moieties in a volume of fluid. The amount of detected light may be compared to a standard measure of known association of light to the number of moieties to determine the number of molecules associated. For example, each fluorescent moiety emits a photon of light in response to an absorbed photon of light in the moieties excitation range (also referred to as the absorption range) where the emitted photon is at a longer wavelength than the wavelength of the excitation photon (generally referred to as a “Stokes Shift”). Thus, the intensity of light emitted from a pool of fluorescent moieties in response to a known intensity of excitation light is based, at least in part, upon the number of fluorescent moieties in the pool. In the present example, a single fluorescent moiety is associated with each embodiment of adaptor 200 or 400, so that each embodiment of adapted nucleic acid 360 comprises two fluorescent moieties. Therefore, there is a direct association of the number of fluorescent moieties to the number of adapted nucleic acid molecules in a sample that is easily measurable using standard excitation sources (i.e. laser, LED, UV, or incandescent sources) and detection devices (i.e. Fluorometer, CCD, or confocal detection architectures) known in the art. The species of fluorescent moiety may include, but is not limited to Cy3, Cy5, carboxyfluorescein (FAM), Alexafluor, Rhodamine green, Texas Red, R-Phycoerytherin, semiconductor nanocrytals (also referred to as “Quantum Dots”), or other fluorescent species known in the art.

An illustrative example of a detectable moiety associated with adaptor 200 is provided in FIG. 2A as detectable moiety 270. As described above, moiety 270 may include a fluorescent moiety, enzymatic conjugates (i.e. alkaline phosphatase or horseradish peroxidase), or other type of detectable moiety known to those of ordinary skill. In preferred embodiments, moiety 270 is positionally located in the non-complementary of Y-region 207 that also contributes to the inhibition of ligation of the end of region 207 with other molecules.

As described above, the positional relationship of adaptors 200′ and 200″ relative to each other in adapted nucleic acid 360 results in each strand of adapted nucleic acid 360 having key components appropriately positioned for downstream processing steps that in some embodiments include amplification primer sites 253 and 255 for increasing the copy number of each strand via PCR or other similar process, and sequencing primer site 260 for determination of the sequence composition of each strand via sequencing methods described above. As illustrated in FIG. 3 due to the directional ligation of adaptor 200 to the ends of nucleic acid target 305, each strand of adapted target nucleic acid 350 comprises an embodiment of amplification primer site 253, amplification primer site 255, and sequencing primer site 260. For example, the strands are dissociated from each other and each are separately amplified to produce clonal libraries amenable for sequencing. Preferably, the clonal amplification is performed using the emPCR methods described herein, resulting in amplified libraries that are sequestered to solid supports. In typical emPCR embodiments an amplification primer species is immobilized upon a bead support and a second primer species is in a reaction solution (i.e. in solution phase) both encapsulated within an aqueous droplet which compartmentalizes the reaction environment. In the present example, the immobilized primer species is complementary to amplification primer site 255 and the solution phase primer is complementary to amplification primer site 253, however those of ordinary skill will appreciate that the alternative combination is also possible.

Continuing the example from above, sequencing primer site 260 is positionally located next to the sequence of target nucleic acid 305 in adapted nucleic acid 360 and amenable for use in sequencing methods that employ a polymerase for synthesis and detection of incorporated nucleic acid species. The relative position of sequencing primer site 260 in adapted nucleic acid 360 is important so that the sequencing real estate is preserved by not generating sequence data from elements of adaptor 200 that are already known. However, in some embodiments there are exceptions where elements are positioned relative to sequencing primer site 260 for the express purpose of producing sequence data from them. The sequence data generated from these elements are subsequently employed for the purposes of quality control, multiplex identification, or other purpose for which the respective element is designed to achieve.

One such element may include a 4 base “Key sequence” element that typically, as described above, serves as a quality control element. Another element that may be included in the same or alternative embodiment includes what is the referred to as a “Multiplex Identifier” (also referred to as an MID). In some embodiments, it is desirable to combine nucleic acid fragments from different samples, individuals, etc. in order to maximize the cost and efficiency of the sequencing process where it becomes necessary to understand the origin of each sequence post processing in order to appreciate the biological and/or diagnostic significance. In preferred embodiments the sequence composition of each MID selected for use in a sequencing process is designed so that a number of sequencing errors that could be introduced into the sequence data generated from an MID element are recognized and correctable. Embodiments of MID's amenable for use with the present invention are described in U.S. patent application Ser. No. 12/156,242, incorporated by reference above.

In some embodiments, MID elements may be specifically adapted to employ with adaptor 200 or 400. However, it will be appreciated that the specialized MID elements are not necessarily required for use with adaptors 200 or 400. For example, the adaptations of the MID elements are implemented in the rules used for their design and detection/correction of errors. A first consideration for MID design and recognition for adaptor 200 is that the first sequence position of the MID should not include the same composition as the neighboring sequence position, and thus if for instance the neighboring sequence position belongs to the key sequence and ends with a T nucleotide species, the MID elements cannot start with a T. A second consideration includes the possible requirement of a specific nucleotide species at the last position in certain embodiments, such as the requirement of the T species in the last position as described above for the sticky ended ligation using the A/T nucleotides species combination. In the present example, it may also be advantageous to employ what may be viewed as a “relaxed” criteria for the design of MID elements for detection and correction possibilities which includes using a minimum edit distance (also sometimes referred to as MED) of 4 that allows for the detection of up to 2 errors with the correction of 1 or the detection of up to 3 errors with the correction of 0 (where #errors_{detect} +#errors_{coffect}+1≦MED). In the present example, the errors may include insertion, deletion, or substitution errors (a substitution error typically counts as one deletion error and one insertion error) as described in the Ser. No. 12/156,242 application described above. The advantage of using the relaxed criteria is that it allows for a larger number of MID elements to be used, especially advantageous if the rate of sequencing errors is known or expected to be low. Continuing with the present example, an MID element may be positioned on a strand of adaptor 200 or 400 immediately adjacent to sequencing primer site 260 or key element as described above. In typical sequencing application, the sequence composition will thus be generated early in the process that limits the degree of introduced error and the positional location known in the resulting sequence composition. The known positional location is important for the association of the MID sequence composition with the sample of origin.

For example, additional considerations were employed to design 133, 11 base pair long MID sequence elements for use with adaptor 200. In the present example, the MID elements described herein include an additional base position than those described in the Ser. No. 12/156,242 application which is included because the last position is always the same (i.e. T) as described above. Further, the MID element is designed so that no more than 24 flows would be required to sequence through the MID element. The MID sequence elements of the present example are illustrated below in Table 1.

TABLE 1
11 bp, Max 24flows

| | | | | SEQ |
|---|---|---|---|---|
| | | | | ID |
| | Flow Signals | Flows | Sequence | NO: |
| CYMid1 | 0111011111111 | 13 | ACGACGTACGT | 1 |
| CYMid2 | 01100111011101101 | 17 | ACACGACGACT | 2 |
| CYMid3 | 01100111110111001 | 17 | ACACGTAGTAT | 3 |
| CYMid4 | 01100110111010111 | 17 | ACACTACTCGT | 4 |
| CYMid5 | 01110110011111001 | 17 | ACGACACGTAT | 5 |
| CYMid6 | 01110101110101101 | 17 | ACGAGTAGACT | 6 |
| CYMid7 | 01110011101011011 | 17 | ACGCGTCTAGT | 7 |
| CYMid8 | 01111110011001101 | 17 | ACGTACACACT | 8 |
| CYMid9 | 01111110100110011 | 17 | ACGTACTGTGT | 9 |
| CYMid10 | 01111101010010111 | 17 | ACGTAGATCGT | 10 |
| CYMid11 | 01101111101010101 | 17 | ACTACGTCTCT | 11 |
| CYMid12 | 01101100111101011 | 17 | ACTATACGAGT | 12 |
| CYMid13 | 01101011001110111 | 17 | ACTCGCGTCGT | 13 |
| CYMid14 | 01010110101101111 | 17 | AGACTCGACGT | 14 |
| CYMid15 | 01011111010101011 | 17 | AGTACGAGAGT | 15 |
| CYMid16 | 01011110111011001 | 17 | AGTACTACTAT | 16 |
| CYMid17 | 01011101011110101 | 17 | AGTAGACGTCT | 17 |
| CYMid18 | 01011011111001101 | 17 | AGTCGTACACT | 18 |
| CYMid19 | 01011001110111011 | 17 | AGTGTAGTAGT | 19 |
| CYMid20 | 01001101110011111 | 17 | ATAGTATACGT | 20 |
| CYMid21 | 00100101111111101 | 17 | CAGTACGTACT | 21 |
| CYMid22 | 00110111011100111 | 17 | CGACGACGCGT | 22 |
| CYMid23 | 00110111010111101 | 17 | CGACGAGTACT | 23 |
| CYMid24 | 00110100111011111 | 17 | CGATACTACGT | 24 |
| CYMid25 | 00111111101101001 | 17 | CGTACGTCGAT | 25 |
| CYMid26 | 00101110101111011 | 17 | CTACTCGTAGT | 26 |
| CYMid27 | 00011110010111111 | 17 | GTACAGTACGT | 27 |
| CYMid28 | 00011011111111001 | 17 | GTCGTACGTAT | 28 |
| CYMid29 | 00011001111101111 | 17 | GTGTACGACGT | 29 |
| CYMid30 | 011001100101100101011 | 21 | ACACAGTGAGT | 30 |
| CYMid31 | 011001101010010011101 | 21 | ACACTCATACT | 31 |
| CYMid32 | 011001010110010100111 | 21 | ACAGACAGCGT | 32 |
| CYMid33 | 011001010110110011001 | 21 | ACAGACTATAT | 33 |
| CYMid34 | 011001010101011010101 | 21 | ACAGAGACTCT | 34 |
| CYMid35 | 011001010010101110011 | 21 | ACAGCTCGTGT | 35 |
| CYMid36 | 011001011001101101001 | 21 | ACAGTGTCGAT | 36 |
| CYMid37 | 011101010011001100101 | 21 | ACGAGCGCGCT | 37 |
| CYMid38 | 011101001001010110011 | 21 | ACGATGAGTGT | 38 |
| CYMid39 | 011100110101010101001 | 21 | ACGCGAGAGAT | 39 |
| CYMid40 | 011100101010101010101 | 21 | ACGCTCTCTCT | 40 |
| CYMid41 | 011110110010100101001 | 21 | ACGTCGCTGAT | 41 |
| CYMid42 | 011110101101001001001 | 21 | ACGTCTAGCAT | 42 |
| CYMid43 | 011011011001010011001 | 21 | ACTAGTGATAT | 43 |
| CYMid44 | 011010100110011010011 | 21 | ACTCACACTGT | 44 |
| CYMid45 | 011010100110110100101 | 21 | ACTCACTAGCT | 45 |
| CYMid46 | 011010101100110011001 | 21 | ACTCTATATAT | 46 |
| CYMid47 | 011010010100101010111 | 21 | ACTGATCTCGT | 47 |
| CYMid48 | 011010010010100111101 | 21 | ACTGCTGTACT | 48 |
| CYMid49 | 011010011101001100101 | 21 | ACTGTAGCGCT | 49 |
| CYMid50 | 010101100110101001101 | 21 | AGACACTCACT | 50 |
| CYMid51 | 010101100100110011011 | 21 | AGACATATAGT | 51 |
| CYMid52 | 010101111001010010101 | 21 | AGACGTGATCT | 52 |
| CYMid53 | 010101011110010101001 | 21 | AGAGTACAGAT | 53 |
| CYMid54 | 010101011100101010101 | 21 | AGAGTATCTCT | 54 |
| CYMid55 | 010101001111001010011 | 21 | AGATACGCTGT | 55 |
| CYMid56 | 010101001010110110101 | 21 | AGATCTAGTCT | 56 |
| CYMid57 | 010100100101001111011 | 21 | AGCAGCGTAGT | 57 |
| CYMid58 | 010100110010011101011 | 21 | AGCGCACGAGT | 58 |
| CYMid59 | 010100111001100100111 | 21 | AGCGTGTGCGT | 59 |
| CYMid60 | 010100101101010011101 | 21 | AGCTAGATACT | 60 |
| CYMid61 | 010100101001101101101 | 21 | AGCTGTCGACT | 61 |
| CYMid62 | 010111001001001001111 | 21 | AGTATGCACGT | 62 |
| CYMid63 | 010110110011001011001 | 21 | AGTCGCGCTAT | 63 |
| CYMid64 | 010110101001101010011 | 21 | AGTCTGTCTGT | 64 |
| CYMid65 | 010011100110011101001 | 21 | ATACACACGAT | 65 |
| CYMid66 | 010011110011100100101 | 21 | ATACGCGTGCT | 66 |
| CYMid67 | 010011101101001001101 | 21 | ATACTAGCACT | 67 |
| CYMid68 | 010011010101001011011 | 21 | ATAGAGCTAGT | 68 |
| CYMid69 | 010011001101010111001 | 21 | ATATAGAGTAT | 69 |
| CYMid70 | 010010110010101001111 | 21 | ATCGCTCACGT | 70 |
| CYMid71 | 010010111010010110101 | 21 | ATCGTCAGTCT | 71 |
| CYMid72 | 010010101010101111001 | 21 | ATCTCTCGTAT | 72 |
| CYMid73 | 010010101001010101111 | 21 | ATCTGAGACGT | 73 |
| CYMid74 | 010010010010111110101 | 21 | ATGCTACGTCT | 74 |
| CYMid75 | 010010011001011011101 | 21 | ATGTGACTACT | 75 |
| CYMid76 | 001001110101011001011 | 21 | CACGAGACAGT | 76 |
| CYMid77 | 001001110011010110101 | 21 | CACGCGAGTCT | 77 |
| CYMid78 | 001001110010111101001 | 21 | CACGCTACGAT | 78 |
| CYMid79 | 001001111001110011001 | 21 | CACGTGTATAT | 79 |
| CYMid80 | 001001101111010010011 | 21 | CACTACGATGT | 80 |
| CYMid81 | 001001101100111010101 | 21 | CACTATACTCT | 81 |
| CYMid82 | 001001010011111010011 | 21 | CAGCGTACTGT | 82 |
| CYMid83 | 001001011010101011011 | 21 | CAGTCTCTAGT | 83 |
| CYMid84 | 001001001101101100111 | 21 | CATAGTCGCGT | 84 |
| CYMid85 | 001101010110011011001 | 21 | CGAGACACTAT | 85 |
| CYMid86 | 001101010101100110011 | 21 | CGAGAGTGTGT | 86 |
| CYMid87 | 001101011010010010111 | 21 | CGAGTCATCGT | 87 |
| CYMid88 | 001101001011110011001 | 21 | CGATCGTATAT | 88 |
| CYMid89 | 001100100101111100101 | 21 | CGCAGTACGCT | 89 |
| CYMid90 | 001100110100101111001 | 21 | CGCGATCGTAT | 90 |
| CYMid91 | 001100110010110011101 | 21 | CGCGCTATACT | 91 |
| CYMid92 | 001111100101010011001 | 21 | CGTACAGATAT | 92 |
| CYMid93 | 001111010010101010101 | 21 | CGTAGCTCTCT | 93 |
| CYMid94 | 001111001101100100101 | 21 | CGTATAGTGCT | 94 |
| CYMid95 | 001110100101001101101 | 21 | CGTCAGCGACT | 95 |
| CYMid96 | 001110110010010110011 | 21 | CGTCGCAGTGT | 96 |
| CYMid97 | 001110101010011101001 | 21 | CGTCTCACGAT | 97 |
| CYMid98 | 001110010110101001011 | 21 | CGTGACTCAGT | 98 |
| CYMid99 | 001011100111001010101 | 21 | CTACACGCTCT | 99 |
| CYMid100 | 001011110100110010011 | 21 | CTACGATATGT | 100 |
| CYMid101 | 001011010110010101101 | 21 | CTAGACAGACT | 101 |
| CYMid102 | 001011011110101001001 | 21 | CTAGTACTCAT | 102 |
| CYMid103 | 001011001100100110111 | 21 | CTATATGTCGT | 103 |
| CYMid104 | 001011001011011001101 | 21 | CTATCGACACT | 104 |
| CYMid105 | 001011001001110101011 | 21 | CTATGTAGAGT | 105 |
| CYMid106 | 001010100111111001001 | 21 | CTCACGTACAT | 106 |
| CYMid107 | 001010110101101010101 | 21 | CTCGAGTCTCT | 107 |
| CYMid108 | 001010111011010101001 | 21 | CTCGTCGAGAT | 108 |
| CYMid109 | 001010101110010100111 | 21 | CTCTACAGCGT | 109 |
| CYMid110 | 001010011011100100111 | 21 | CTGTCGTGCGT | 110 |
| CYMid111 | 001010011001011110011 | 21 | CTGTGACGTGT | 111 |
| CYMid112 | 000101110010100110111 | 21 | GACGCTGTCGT | 112 |
| CYMid113 | 000101111100100101101 | 21 | GACGTATGACT | 113 |
| CYMid114 | 000101101101001011011 | 21 | GACTAGCTAGT | 114 |
| CYMid115 | 000101010111101100101 | 21 | GAGACGTCGCT | 115 |
| CYMid116 | 000101010101010101111 | 21 | GAGAGAGACGT | 116 |
| CYMid117 | 000100111101011011001 | 21 | GCGTAGACTAT | 117 |
| CYMid118 | 000100111011100110101 | 21 | GCGTCGTGTCT | 118 |
| CYMid119 | 000100101010101011111 | 21 | GCTCTCTACGT | 119 |
| CYMid120 | 000111100110100111001 | 21 | GTACACTGTAT | 120 |
| CYMid121 | 000111110011011001001 | 21 | GTACGCGACAT | 121 |
| CYMid122 | 000111101100110101001 | 21 | GTACTATAGAT | 122 |
| CYMid123 | 000111101001010110101 | 21 | GTACTGAGTCT | 123 |
| CYMid124 | 000111010010110100111 | 21 | GTAGCTAGCGT | 124 |
| CYMid125 | 000111011010011010011 | 21 | GTAGTCACTGT | 125 |
| CYMid126 | 000111011001101001101 | 21 | GTAGTGTCACT | 126 |
| CYMid127 | 000111001110010011011 | 21 | GTATACATAGT | 127 |
| CYMid128 | 000110100100101110111 | 21 | GTCATCGTCGT | 128 |
| CYMid129 | 000110110110011100101 | 21 | GTCGACACGCT | 129 |
| CYMid130 | 000110110101100101011 | 21 | GTCGAGTGAGT | 130 |
| CYMid131 | 000110101110110010101 | 21 | GTCTACTATCT | 131 |
| CYMid132 | 000110011010110101101 | 21 | GTGTCTAGACT | 132 |
| CYMid133 | 000110011001110010111 | 21 | GTGTGTATCGT | 133 |

As described above, processing adapted nucleic acid 350 for sequencing includes a dissociation step that separates the strands which in some embodiments may be sequenced directly. In other embodiments it is desirable to individually amplify each strand to produce a clonal library of substantially identical copies, which may, in some embodiments be sequestered to a solid support or otherwise compartmentalized to maintain the uniformity of the clonal population. As described above, a very efficient method for producing clonal libraries includes the emPCR method where each template strand is introduced into an aqueous emulsion droplet comprising a bead with an immobilized primer species and all reagents necessary to carry out a PCR amplification reaction. In embodiments that employ clonal amplification, such as PCR, it can be desirable to incorporate additional design elements into the adaptor of the invention to improve amplification efficiency.

One problem that can occur during thermocycling steps of PCR type amplification processes is that the ends of the adapted single stranded template can anneal due to the complementary nature of the sequence composition in the adaptor regions at the ends forming what are referred to as hairpin structures. For example, FIG. 3 provides an illustrative representation of adapted nucleic acid 350 comprising strands 311 and 313 each including an embodiment of amplification primer site 253 coupled with sequencing primer site 260 at one adapted end and site 363 coupled with amplification primer site 255 at the other adapted end. It will be appreciated by those of ordinary skill that amplification primer sites 253 and 255 are complementary to each other and that sequencing primer site 260 is complementary to site 363. Further it will be understood that the positional arrangements of the complementary sites at each end can promote the formation of hairpin structures. Such hairpin structures have an inhibitory effect on typical PCR amplification process, due at least in part to the inability of the polymerase to read through the annealed region of the hairpin. Also, the region of adapted nucleic acid comprising nucleic acid target 305 may include secondary structure that further adds stability to the hairpin structure, which may increase as GC content increases, which further reduces the likelihood of successful amplification. In addition, as the copy number increases in the rounds of amplification (i.e. rounds of alternating thermocycling between a denaturation temperature and an annealing temperature) the likelihood of some percentage of the amplified copies forming hairpin structures increases. It will also be appreciated that the likelihood further increases as the GC content of the adaptor regions increases due to the stronger base pairing relationship of G and C nucleotide species, resulting in what may be referred to as a “GC bias”. Thus, it is desirable in certain situations to incorporate design elements into the adaptor of the invention that inhibit the formation of hairpin structures.

A useful strategy for reducing the likelihood of hairpin formation includes the incorporation of deoxyinosine species into the design of stem region 205. Those of ordinary skill in the art will appreciate that inosine is a nucleoside species generally considered to be a “universal base” that has the ability to pair with adenine (A), thymine (T), or cytosine (C), and is replaced with a guanine (G) species in the amplified copy by the polymerase. Therefore, the strategy for design includes placing one or more deoxyinosine species on a strand in a base pairing relationship with and A, G, or T, nucleotide species on the complementary strand, typically in stem region 205 so that the amplified copies have a G nucleotide species at the same base position that does not bind to the nucleotide species at that position on the other strand (i.e. the A, G, or T species). The result is a reduced likelihood of the adaptor regions of the amplified copies annealing to one another producing the hairpin structures. Another benefit also includes a reduced likelihood of annealing of separate strands in the inosine-adaptor regions in the amplified copies due to the reduced complementarity with the incorporated G species.

FIG. 4 provides an illustrative example of one embodiment of adaptor 400 comprising inosine 420 at one or more base positions. In the present example, it is desirable that inosine 420 is positioned no closer than six base positions from the end of strand 413. It may be further desirable in the same or alternative embodiments that each implementation of inosine 420 be located no closer than four base positions from each other to prevent re-annealing, where a regular spacing of four or five positions is desirable. Further, the incorporation of inosine 420 into adaptor 400 does not cause significant destabilization of adaptor 400, particularly if the number of inosine 420 embodiments is low relative to the number of base positions in the stem region. Also it is desirable to have a plurality of inosine species in the stem region, where for instance the incorporation of 2 or more inosine species for every 10 bases produces desirable performance. In the example of adaptor 400, the embodiments of inosine 420 are associated with strand 413, however it will be appreciated that embodiments of inosine 420 may be associated with strand 411, or some combination of strands 411 and 413. One important consideration in the selection of strand for inosine incorporation is the composition of elements in the selected strand. For instance, it is desirable to avoid incorporating inosine species into regions used as primers in order to avoid possible weak base paring interactions attributable to the inosine species.

Further, some embodiments of adaptor 200 or 400 are amenable for use in what are generally referred to as “methylation” studies. Those of ordinary skill in the related art appreciate that nucleic acid methylation is involved in developmental processes and cancer and is an important regulatory mechanism for gene expression, where elements associated with methylated promoter regions typically will not be transcribed. In many organisms methylation is associated with CpG sites where DNA methyltyransferase catalyzes the conversion of cytosine to 5-methylcytosine. Nucleic acid sequencing provides a useful tool for studying methylation sites using various techniques. For example, on useful technique is generally referred to as “Bisulfite” treatment that changes the nucleic acid composition of a molecule by transforming non-methylated cytosine residues to Uracil. The bisulfite treated nucleic acid molecules may then be sequenced and the sites of methylation identified. In the present example, embodiments of adaptor 200 or 400 may be methylated to protect the C nucleotide species from the bisulfite, and associated with the subject nucleic acid molecules as described herein.

Also described above, the adaptors of the invention operate cooperatively with complementary technologies, such as microarray technologies. For example, embodiments of adaptor 200 or 400 are amenable for use with specialized microarray technology such as what is referred to as “Sequence Capture” type microarray technology that is capable of selectively capturing nucleic acid molecules of interest and releasing the selected pool for additional analysis (generally described in Albert et al. Nature Methods published online Oct. 14, 2007: Direct selection of human genomic loci by microarray hybridization, which is hereby incorporated by reference herein in its entirety for all purposes). In general sequence capture microarrays comprise a plurality of “capture probes” designed to bind to specific nucleic acid target sequences under conditions that favor hybridization. Embodiments of sequence capture microarray may differ in the density and/or number of capture probes disposed upon the array substrate, but may include at least 10,000 capture probes, at least 100,000 capture probes, at least 1,000,000 capture probes, or other number of capture probes enabled by the microarray manufacturing technology and desired application. This is especially useful for sequencing the selected pool of nucleic acid molecules. In the present example, it is sometimes desirable to optimize sequencing resources for reasons of efficiency such as cost (i.e. reagent usage, facility costs, etc.), time (i.e. technician time, instrument time, etc.). It is also desirable in such circumstances to focus the data processing to only nucleic acid molecules of interest. It is clear to one skilled in the art that an important aspect of Sequence Capture technology is hybridization mediated complexity reduction. Whether the hybridization that is the basis for the molecular enrichment happens upon a solid support such as a microarray, or in the liquid phase (i.e. capture probes liberated from a solid support) does not matter for employment in this embodiment. Additional examples of sequence capture microarray technology are provided in U.S. patent application Ser. Nos. 11/789,135 and 11/970,949 incorporated by reference above.

In addition, the use of microarray sequence capture technology with embodiments of adaptor 200 or 400 derives additional benefits from adaptor embodiments that comprise embodiments of the MID elements described above. For example, as described above the MID elements enable the pooling of nucleic acid molecules from different samples and sequencing where the sequence composition of the MID element(s) can be used to associate the sequence with the original sample. In some embodiments it is further advantageous to combine this strategy with the microarray sequence capture technology because the advantages conferred by each are complementary and provide a powerful and cost effective method for analysis of specific sequence information of interest from different samples (i.e. from individuals, tissues, cultures, or other source generally known in the related art). Thus, allowing for comparison of the targeted sequence information between the different samples. Additional examples of sequence capture using MID adapted is described in U.S. Provisional Patent Application Ser. No. 61/032,149, titled “Methods and Systems for Multiplexed Nucleic Acid Sequence

Analysis”, filed Feb. 28, 2008, which is hereby incorporated by reference herein in its entirety for all purposes.

### Examples

1) Nucleic Acid Preparation and Fluorescent Quantification
1. DNA Fragmentation via Nebulization −20 psi vented nebulizer
2. Minelute column
3. SPRI size exclusion to narrow library distribution
   1) 0.50:1 SPRI to product and collect non-bound supernatant
   2) 0.65:1 SPRI to Product and collect eluate from Beads
4. Polishing Reaction (22 C for 20 minutes)
   1) 23 ul of sample in 1×TE
   2) 5 ul Polishing Buffer (454 kit)
   3) 5 ul BSA (454 kit)
   4) 5 ul ATP (454 kit)
   5) 2 ul dNTP (454 kit)
   6) 5 ul T4 PNK (454 kit)
   7) 5 ul T4 DNA polymerase (454 kit)
5. Minelute column
6. Ligation Reaction (22 C for 10 minutes)
   1) 14 ul of polished sample in 1×TE
   2) 20 ul of Ligation Buffer (454 kit)
   3) 2 ul of FAM adaptor at 50 micromolare
   4) 4 ul of Ligase (454 kit)
7. Qiaquick column with 8M guanidine HCl wash after binding and before PE wash
8. SPRI Size Exclusion at 0.65:1 SPRI beads to product to remove adaptor dimmers
9. Quantify using the blue filter on a TBS-380 flourometer and using the previously quantified FAM oligo as the standard.
   Heat Denature to single strand the DNA

2) Inosine Incorporation and Comparison of Binding Energy

Adaptors were designed with and without inosine nucleotides and a comparison of the relative binding energy of the amplified products to their complements and amplification efficiency was made.

The first adaptor designed without inosine included the following composition with the top strand representing pre amplification sequence composition and the bottom representing post amplification sequence composition. The resulting binding energy was a ΔG of −25.71 kcal/mole.

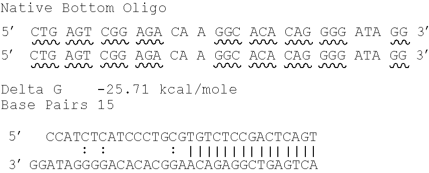

(SEQ ID NOS 134 and 134-136, respectively, in order of appearance)

The second adaptor designed to include inosine included the following composition with the top strand representing pre amplification sequence composition and the bottom representing post amplification sequence composition. The resulting binding energy was a ΔG of −9.41 kcal/mole.

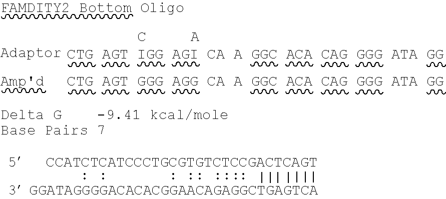

(SEQ ID NOS 137-138, 135 and 139, respectively, in order of appearance)

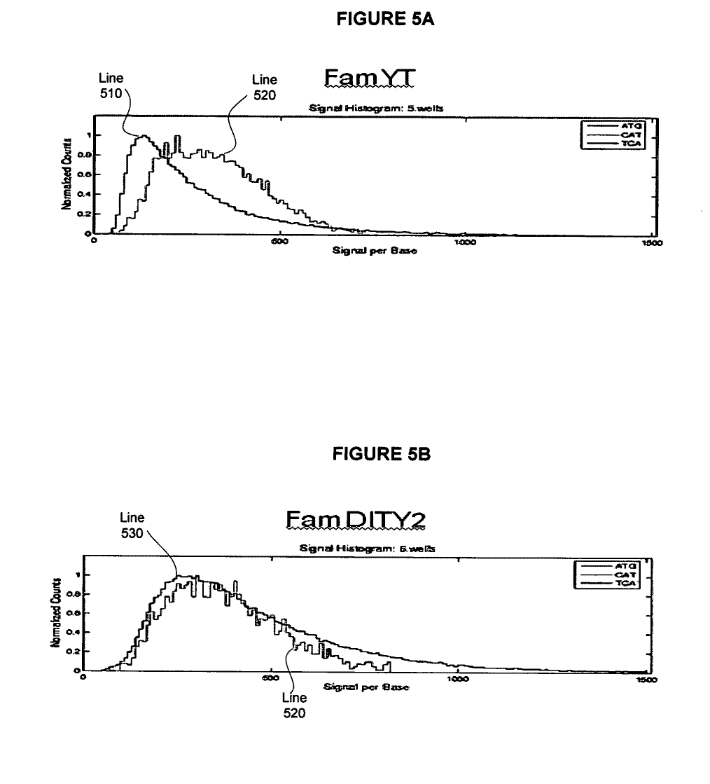

FIGS. 5A and 5B illustrate the difference in amplification efficiency between an embodiment of adaptor the comprising inosine and an embodiment of adaptor lacking inosine. The results were obtained from sequencing libraries made from T. thermophilus which contains a genome that comprises about 70% GC content using the two different adaptor compositions.

Line 510 in FIG. 5A shows the result of inefficient amplification produced from sequencing 5 reaction wells using the non-inosine adapted library comprising the “native bottom oligo” composition represented above. Those of ordinary skill will appreciate the there is a substantial drop-off in the detected “signal per base” as the sequence length increases. This is in contrast to line 520 that illustrates detected signals from a population of “test fragments” of known composition and length to provide an internal control for the performance of the sequencing process. If the adapted library amplified efficiently lines 510 and 520 should have similar distributions as they do in FIG. 5B.

Line 530 in FIG. 5B shows the detected signals produced from sequencing 5 reaction wells using a library amplified using the “FamDITY2_Bottom Oligo”. It will be appreciated that lines 530 and 520 have similar distribution patterns that show that that the adaptors comprising inosine amplified efficiently producing comparable results to the known population represented by line 520.

3) Sequence Capture and Sequencing of Two Combined MID Y Adapted DNA Libraries

Two separate MID-adapter tagged libraries were created; sample NA04671 (Burkitt's Lymphoma cell line, CORIELL Institute for Medical Research, Camden N.J.) was adapted with MID1 adapter molecules, while sample NA11839 (CEPH/Utah Pedigree 1349, CORIELL Institute for Medical Research) was tagged with MID6 adapters. The two MID-tagged libraries were pooled and co-hybridized to a sequence capture microarray designed with probes targeting loci of cumulative size ˜228 Kbp on human chromosome 8q24. The eluate was collected, amplified by Ligation Mediated PCR (LM-PCR), and then emPCR, and subjected to 454 sequencing. Sequencing yielded approximately 225,619 reads comprising 47,380,626 base pairs.

Standard 454 base-calling and trimming procedures were applied to yield high-quality sequence and quality files. Each read was aligned to each MID tag used in order to determine whether a read combined one or more of the tags. Reads with one uniquely identifiable tag were retained, while those with no tag, more than one unique tag (>=1 copies each of MID1 and MID6) or more than one copy of a tag (>=1 copies of MID1) were rejected (Table 2). The majority of reads contained exactly one MID tag, identifying their sample of origin. As seen in Table 2, the MID6- NA11839 library species is approximately 3.7-fold over-represented, suggesting that adapted libraries were pooled in unequal proportions, but consistent with pipetting error, or a difference in the efficiency in ligation of that MID over the other sample understudy.

The MID tags were trimmed from passed reads, which were then mapped to the human genome assembly (NCBI build 36.1) using NCBI MegaBLAST. Reads with no hit to the genome, and with multiple hits amongst which a single best hit could not be distinguished were discarded. Following alignment, 33842 (80.4%) of MID1-tagged reads and 127050 (82.8%) of MID6-tagged reads mapped uniquely to the genome. Comparing reads' mapped coordinates to the targeted interval, 3185 (7.6%) of MID1-tagged reads and 12252 (8.0%) of MID6-tagged reads mapped to within the target region, representing simultaneous fold-enrichment values of 1033× and 1087×, respectively.

TABLE 2
Read counts categorized by MID tag presence.

| MID tag call | Number of reads | Percentage of reads |
|---|---|---|
| Passed: MID1 | 42080 | 18.6% |
| Passed: MID6 | 153533 | 68.0% |
| Rejected: Both MID1 and MID6 | 4259 | 1.9% |
| Rejected: >1 copy, MID1 and/or | 16280 | 7.2% |
| MID6 | | |
| Rejected: No tag found | 9533 | 4.2% |

Having described various embodiments and implementations, it should be apparent to those skilled in the relevant art that the foregoing is illustrative only and not limiting, having been presented by way of example only. Many other schemes for distributing functions among the various functional elements of the illustrated embodiment are possible. The functions of any element may be carried out in various ways in alternative embodiments.

What is claimed is:

1. An adaptor element for efficient target processing, comprising:
a semi-complementary double stranded nucleic acid adaptor comprising a non-complementary region and a complementary region, wherein the non-complementary region comprises a first amplification primer site and a second amplification primer site and the complementary region comprises a sequencing primer site and one or more inosine species.

2. The adaptor element of claim 1, wherein:
the non-complementary region comprises a detectable moiety.

3. The adaptor element of claim 2, wherein:
the detectable moiety comprises a fluorescent label.

4. The adaptor element of claim 3, wherein:
the fluorescent label is selected from the group consisting of Cy3, Cy5, carboxyfluorescein (FAM), Alexafluor, Rhodamine green, Texas Red, R-Phycoerytherin, semiconductor nanocrytals.

5. The adaptor element of claim 1, wherein:
the complementary region comprises a blunt end.

6. The adaptor element of claim 5, wherein:
The blunt end is ligatable to a blunt end of a target nucleic acid.

7. The adaptor element of claim 1, wherein:
the complementary region comprises a sticky end.

8. The adaptor element of claim 7, wherein:
the sticky end comprises a single base overhang.

9. The adaptor element of claim 8, wherein:
the single base overhang comprises a T nucleotide species.

10. The adaptor element of claim 7, wherein:
the sticky end comprises an overhang comprising a plurality a bases.

11. The adaptor element of claim 1, wherein:
the complementary region comprises a multiplex identifier element.

12. The adaptor element of claim 11, wherein:
the multiplex identifier element comprises 11 sequence postions.

13. The adaptor element of claim 12, wherein:
the multiplex identifier element is selected from the group consisting of SEQ ID NO 1-SEQ ID NO 133.

14. The adaptor element of claim 11, wherein:
the multiplex identifier element comprises a design that enables detection of up to two sequencing errors and correction of one of the sequencing errors.

15. The adaptor element of claim 1, wherein:
the inosine species are positionally located in a single strand.

16. The adaptor element of claim 15, wherein:
the inosine species are positionally located at least four sequence positions from the end of the strand.

17. The adaptor element of claim 15, wherein:
at least two of the inosine species are positionally located no closer than four sequence positions from each other.

18. The adaptor element of claim 1, wherein:
the complementary region comprises one or more phosphorothioate species.

19. The adaptor element of claim 18, wherein:
the non-complementary region comprises one or more phosphorothioate species.

20. The adaptor element of claim 19, wherein:
the phosphorothioate species are positionally located in an end region of the complementary and non-complementary regions.

21. The adaptor element of claim 18, wherein:
the phosphorothioate species protect the end regions from exonuclease digestion.

22. A kit comprising:
the semi-complementary double stranded nucleic acid adaptor of claim 1.

23. A method for efficient target processing, comprising:
ligating a species of a double stranded nucleic acid adaptor to each end of a linear double stranded nucleic acid molecule to produce an adapted double stranded nucleic acid molecule, wherein the species of the double stranded nucleic acid adaptor comprises a complementary region amenable for ligation to the linear double stranded nucleic acid molecule and a non-complementary region that inhibits ligation;
dissociating the adapted double stranded nucleic acid molecule to produce a first strand and a second strand each comprising a first amplification primer site and a sequencing primer site at a first end and a second amplification site at a second end; and
individually amplifying the first and second strands to produce a first clonal population comprising copies of the first strand and a second clonal population comprising copies of the second strand.

24. The method of claim 23, further comprising:
sequencing the first clonal population to produce a sequence composition of the first strand.

25. The method of claim 24, further comprising:
associating the sequence composition with a sample of origin, wherein the sequence composition comprises a sequence from a multiplex identifier element included in the double stranded nucleic acid adaptor.

26. The method of claim 25, wherein:
the multiplex identifier element comprises 11 sequence postions.

27. The method of claim 26, wherein:
the multiplex identifier element is selected from the group consisting of SEQ ID NO 1-SEQ ID NO 133.

28. The method of claim 25, wherein:
the step of associating comprises detection of up to two errors in the sequence from the multiplex identifier element and correction of up to one of the sequencing errors.

29. The method of claim 23, further comprising:
prior to the step of dissociating, determining a quantity of the adapted double stranded nucleic acid, wherein the double stranded nucleic acid adaptor comprises a fluorescent moiety.

30. The method of claim 29, further comprising:
the fluorescent moiety emits light in response to an excitation light and is measured by a detector, wherein a level of the measured emitted light is associated with the quantity.

31. The method of claim 29, further comprising:
the fluorescent moiety is selected from the group consisting of Cy3, Cy5, carboxyfluorescein (FAM), Alexafluor, Rhodamine green, Texas Red, R-Phycoerytherin, semiconductor nanocrytals.

32. The method of claim 23
the complementary region comprises one or more inosine species.

33. The method of claim 32, wherein:
the inosine species are positionally located in a single strand.

34. The element of claim 33, wherein:
the inosine species are positionally located at least six sequence positions from the end of the strand.

35. The element of claim 33, wherein:
at least two of the inosine species are positionally located no closer than four sequence positions from each other.

36. The element of claim 33, wherein:
the inosine species inhibit the formation of hairpin structures of the first strand and the second strand.

37. The element of claim 33, wherein:
the inosine species improve amplification efficiency of the first strand and the second strand.

38. A method for multiplex target processing and enrichment, comprising:
ligating a species of a double stranded nucleic acid adaptor to each end of a plurality of linear double stranded nucleic acid molecules from a plurality of samples to produce a pool of adapted double stranded nucleic acid molecules, wherein the species of the double stranded nucleic acid adaptor comprises a sample specific identifier element;
dissociating a plurality of members from the pool adapted double stranded nucleic acid molecules to produce a first strand and a second strand from each of the dissociated members to produce a population of single stranded molecules;
hybridizing a plurality of members of the population of single stranded molecules to a substrate bound capture probe, wherein the population of single stranded molecules comprises at least one member that does not hybridize to a substrate bound capture probe;
eluting the hybridized members from the substrate bound capture probe to produce an enriched population of single stranded molecules;
amplifying a plurality of members of the enriched population of single stranded molecules to produce a clonal population from each amplified member;
individually sequencing the clonal populations to produce sequence data for each amplified member that comprises a sequence composition for the multiplex identifier element;
associating the sequence data with one of the samples using the sample specific identifier.
